(19) United States
(12) Patent Application Publication
(10) Pub. No.: US 2026/0364158 A1
(43) Pub. Date: Nov. 19, 2026

(54) HEROIN VACCINE COMPOSITIONS AND METHODS THEREOF

(71) Applicants: Marco PRAVETONI, Minneapolis, MN (US); Michael Dennis RALEIGH, Minneapolis, MN (US); Brian H. LEY, Minneapolis, MN (US); Regents of the University of Minnesota, Minneapolis, MN (US); University of Washington Through Its Center For Commercialization, Seattle, WA (US)

(72) Inventors: Marco Pravetoni, Seattle, WA (US); Michael Dennis Raleigh, Minneapolis, MN (US); Brian H. Heasley, Minneapolis, MN (US)

(73) Assignee: Regents of the University of Minnesota, Minneapolis, MN (US)

(21) Appl. No.: 19/484,698

(22) PCT Filed: May 16, 2024
(86) PCT No.: PCT/US2024/029740

§ 371 Date: Nov. 14, 2025

Related U.S. Application Data
(60) Provisional application No. 63/467,217, filed on May 17, 2023.

(51) Int. Cl. A61K 39/00; A61K 39/39; A61P 37/04; C07D 489/02

(52) U.S. Cl. CPC A61K 39/0013; A61K 39/39; A61P 37/04; C07D 489/02; A61K 2039/55505; A61K 2039/55511; A61K 2039/6081

(57) ABSTRACT

The present disclosure provides vaccine compositions that induce opioid specific antibodies.

### CROSS REFERENCE TO RELATED APPLICATION

This application claims priority to U.S. Provisional Application No. 63/467,217 filed on 17 May 2023, which is incorporated by reference herein.

### GOVERNMENT FUNDING

This invention was made with government support under DA047711, and 75N93020C00039 awarded by the National Institutes of Health. The government has certain rights in the invention.

### BACKGROUND OF THE INVENTION

Current treatment of heroin-use disorder (HUD) uses pharmacotherapies including agonists (methadone, buprenorphine) or antagonists (naltrexone) to bind to opioid receptors in brain to reduce cravings and reinforcing effects of heroin. While current pharmacotherapies are effective, fewer than 25% of patients receive these medications. Additionally, current pharmacotherapies have limited clinical efficacy in preventing fatal overdoses. New prevention or treatment that could complement pharmacotherapies are needed to reduce the incidence of HUD and overdoses involving heroin.

### SUMMARY OF THE INVENTION

Certain embodiments of the invention provide a compound as described herein (e.g., compound 2 or compound 3 in Example 10).

Certain embodiments of the invention provide a conjugate as described herein.

Certain embodiments of the invention provide a vaccine composition comprising one or more adjuvants (e.g., two adjuvants), and a morphine-carrier protein conjugate having structure of Formula I.

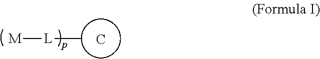

wherein
L is a linker,
C is a carrier protein,
p is a hapten to carrier protein ratio number that is >=1, and
M has structure of

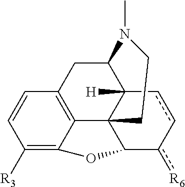

wherein R₃ is OH and R₆ is O or N that is bonded with L; or
   R₆ is OH and R₃ is O that is bonded with L.

Certain embodiments of the invention provide a method of immunizing a subject (e.g., for treatment or prevention of opioid overdose) comprising administering a vaccine composition described herein to the subject.

Certain embodiments of the invention provide a method of treating opioid use disorder (OUD) comprising administering a vaccine composition described herein to the subject.

Certain embodiments of the invention provide a conjugate or composition described herein for the prophylactic or therapeutic treatment of opioid use disorder (OUD) and/or overdose.

Certain embodiments of the invention provide a conjugate or composition described herein for the prophylactic treatment and prevention of opioid fatal and non-fatal overdoses involving heroin alone, or heroin admixed with other opioid and non-opioid compounds.

Certain embodiments of the invention provide a conjugate or composition described herein for use in medical therapy.

Certain embodiments of the invention provide a method for treating opioid use disorder (OUD) in a subject, comprising administering an effective amount of a conjugate or composition described herein to the subject.

Certain embodiments of the invention provide the use of a conjugate or composition described herein to prepare a medicament for the treatment of opioid use disorder (OUD) and/or prevention of overdose in a subject.

Certain embodiments of the invention provide the use of a conjugate or composition described herein to prepare a medicament for the treatment of opioid use disorder (OUD) in a subject already in treatment with current pharmacotherapies (e.g., methadone, buprenorphine, naltrexone).

Certain embodiments of the invention provide the use of a conjugate or composition described herein to prepare a medicament for prevention of toxicity associated with opioid overdose (e.g., respiratory depression) in a subject.

Certain embodiments of the invention provide the use of a conjugate or composition described herein to prepare a medicament for prevention of toxicity associated with opioid overdose (e.g., respiratory depression) in a subject, as a standalone treatment or in combination with approved medications for OUD or overdose reversal (e.g., naloxone).

Certain embodiments of the invention provide a kit comprising a conjugate or composition described herein, packaging material, and instructions for administering the conjugate or composition to a subject to treat opioid use disorder (OUD) and/or to prevent opioid fatal and non-fatal overdose.

Certain embodiments of the invention provide a method, comprising converting morphine (compound 1) to (4R,4aR,7S,7aR,12bS)-9-((4-methoxybenzyl)oxy)-3-methyl-2,3,4,4a,7,7a-hexahydro-1H-4,12-methanobenzofuro[3,2-e]isoquinolin-7-ol (compound 2).

The invention also provides processes and intermediates (e.g., compound 2 or compound 3 in Example 10) disclosed herein that are useful for preparing a compound, or conjugate described herein, as well as compositions described herein.

### BRIEF DESCRIPTION OF THE FIGURES

FIG. 1. Efficacy of heroin vaccine mixed with Tris buffer or INI-4001. Rats (n=8/group) were vaccinated on days 0, 14, 28, and 42. Verification lot (Ver.) was compared to Conformance lot (Conf.) with DP1 and DP2 material. DP2 is 2× concentrated vaccine for ease of mixing 1:1 with INI-4001 adjuvant. ANOVA showed a main effect, but no differences were seen between groups. INI-4001 showed a trend for increased antibody levels. Data are GMT SD.

FIGS. 2A-2D. Efficacy of heroin vaccine mixed with Tris buffer or INI-4001. Rats that received aluminum or DP2 from FIG. 1 were given doses of heroin (from 0.5 up to a cumulative dose of 4 mg/kg, s.c.) every 17 minutes. Antinociception (FIG. 2A), oxygen saturation (FIG. 2B), heart rate (FIG. 2C), and breath rate (FIG. 2D) were measured 15 minutes after every dose. Naloxone (0.1 mg/kg s.c.) was given following the final heroin dose. Data are Mean±SD.

FIGS. 3A-3H. INI-4001 increases the binding and selectivity of heroin vaccine-generated serum antibodies. One week following hotplate and pharmacological measurements from FIG. 2, rats received a single dose of 1 mg/kg heroin s.c., and blood and brain collected 5 minutes later. Serum and brain levels of total opioids (FIGS. 3A, E) in INI-4001 containing groups showed increased efficacy. Individual levels of heroin and its metabolites (6-acetylmorphine and morphine) are shown in panels FIGS. 3B, C, D, F, G, and H. A significant reduction in brain heroin levels were seen in the verification run samples containing INI-4001 compared to Tris buffer. However, the large number of groups reduced the analysis power. Statistical analysis was one-way ANOVA with Dunnett's post test. Data are Mean±SD.

FIGS. 4A-4G. Addition of INI-4001 increases the efficacy of Mor(Gly)4-C₆-sKLH after heroin challenge, and does not change antibody selectivity to methadone. Rats (n=6/group) were immunized on days 0, 21, and 42. Two weeks after final immunization, rats were challenged with 4.5 mg/kg cumulative dose of methadone and (FIG. 4A) latency to respond, (FIG. 4B) oxygen saturation, and (FIG. 4C) heart rate were measured 15 minutes after each dose. Naloxone was administered 15 minutes after the final dose. The following week, rats were challenged with 4 mg/kg cumulative dose of heroin and (FIG. 4D) latency to respond, (FIG. 4E) oxygen saturation, and (FIG. 4F) heart rate were measured 15 minutes after each dose. (FIG. 4G) Hot plate data expressed as a percent maximum possible effect (% MPE). EC₅₀ was assessed using the dose-response curve for each group. The C₆ hapten was used in this experiment. Data are mean±SEM. Statistical analysis performed via two-way ANOVA with Tukey's multiple comparison's post hoc test. Stars below symbols indicate significance compared to control. Statistical symbols: * p<0.05, **** p<0.0001.

FIGS. 5A-5C. Addition of INI-4001 increases morphine-specific antibody titers and changes pharmacokinetics of heroin metabolite 6-AM. Rats (n=6) were immunized on days 0, 21, and 42. One week after final immunization, blood was collected to analyze (FIG. 5A) morphine-specific serum IgG titers via ELISA. Data are GMT±SD. After heroin challenge, as described in FIG. 5, (FIG. 5B) serum and (FIG. 5C) brain were collected 15 minutes after final heroin dose to measure concentration of 6-AM. The C₆ hapten was used in this experiment. Data are GMT±SD (FIG. 5A) and mean±SEM (FIGS. 5B and C). Statistical analyses performed were unpaired t test with Welch's correction (FIG. 5A) and one-way ANOVA followed by Tukey's multiple comparisons post hoc test (FIGS. 5B and C). Statistical symbols: ** p<0.01, *** p<0.001, **** p<0.0001.

FIGS. 6A-6D. Efficacy of M-sKLH with low and high INI-4001 doses. Rats (n=6/group) were immunized on days 0, 21, and 42. Two weeks after final immunization, rats were challenged with 3 mg/kg SC heroin and (FIG. 6A) latency to respond, (FIG. 6B) oxygen saturation, and (FIG. 6C) heart rate were measured 15 and 30 minutes after dosing. Data are mean±SEM. Morphine-specific antibody titers were measured via ELISA (FIG. 6D), with data represented as GMT±SD. The C6 hapten was used in this experiment. Statistical analysis performed via mixed-effects ANOVA with Tukey's multiple comparison's post hoc test. *p<0.05, **p<0.01, ***p<0.001 compared to sKLH control. #p<0.05, ##p<0.01 compared to M-sKLH+alum alone group.

FIG. 7. Experimental design of the toxicology study. Gottingen minipigs (n=46) were randomized and placed into 5 groups (5 males and 5 females). Group 1 received saline, group 2 received a high dose of INI-4001 (75 ug), group 3 received the Low Dose Adjuvanted Heroin Conjugate Vaccine which comprises 40 ug M-sKLH, 60 ug aluminum adjuvant, and 7.5 ug INI-4001, group 4 received the High Dose Adjuvanted Heroin Conjugate Vaccine which comprises 400 ug M-sKLH, 600 ug aluminum adjuvant, and 75 ug INI-4001, and group 5 received the High Dose M-sKLH Conjugate Vaccine, Adsorbed which comprises 400 ug M-sKLH and 600 ug aluminum adjuvant. Groups were vaccinated on days 1, 15, 29, and 43. On Day 50, 3 males and 3 females from each group were sacrificed and blood collected for antibody titer analysis. On Day 78, the remaining minipigs from each group were sacrificed and blood collected for antibody titer analysis.

FIG. 8. INI-4001 increases the morphine-specific heroin vaccine-generated serum antibody titers. Morphine-specific antibody titers did not differ between groups and were combined to show a geometric mean titer (GMT) of 39. All rats had background level titers, indicating no prior exposure to Mor(Gly)4-sKLH conjugate vaccine. At Day 50, group 2 demonstrated that INI-4001 did not induce morphine-specific antibody titers in the absence of vaccine. All minipigs in Group 3 developed significant morphine-specific IgG antibody titers and were consistent with minipigs vaccinated with a low dose of vaccine. Group 4 had GMT that were significantly higher than control, INI-4001, and the low dose vaccination group. Group 5 developed high antibody titers, albeit significantly lower than in minipigs that received the high dose of vaccine and INI-4001. Antibody titers in Group 4 were 86% higher than in Group 5, although this effect was not significant. Antibody titers on Day 78 were 28% lower in Group 4 and 15% lower in Group 5, suggesting that the half-life of morphine-specific antibodies in minipigs are greater than one month. Statistical analysis was one-way ANOVA with Dunnett's post test. Data are Mean±SD.

FIG. 9. Morphine-specific antibody titers were assessed between males (left column) and females (right column) in all groups. No differences were observed between any groups, including INI-4001. Statistical analysis was one-way ANOVA with Dunnett's post test. Data are geometric mean titer±SD.

FIGS. 10A-10B. Effects of M-sKLH+4001 alone or during methadone maintenance on Heroin Self-Administration (HSA) in rats. Mean (±SEM) heroin infusions during baseline and the initial course of four vaccinations (N=4-7 per group). FIG. 10A. Infusions in groups of rats receiving the indicated vaccine or saline, as well as concurrent continuous infusion of sterile water via osmotic minipump during the third and fourth vaccination. FIG. 10B. Mean (±SEM) infusions in groups of rats receiving the indicated vaccine or saline, and either the indicated continuous water or methadone infusion during the third and fourth vaccination.

### DETAILED DESCRIPTION

Certain embodiments of the invention provide a conjugate described herein. In certain embodiments, the conjugate is a morphine hapten-carrier protein conjugate.

In certain embodiments, the conjugate has structure of Formula I.

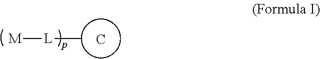

wherein M is morphine, L is a linker, C is a carrier protein, and
p is hapten to carrier protein ratio number that is >=1 (e.g., 1-50, 1-30, or 1-10).

In certain embodiments, p (i.e., hapten number per carrier protein) is about 1-600, 1-500, 1-400, 1-300, 1-200, 1-100, 1-50, 1-40, 1-30, 1-20, or 1-10. In certain embodiments, p is about 10-50, 10-40, 10-30, or 10-20. In certain embodiments, p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or higher number. In certain embodiments, p is at least 1, 2, 3, 4, 5, 6, 7, 8, 9, or 10.

In certain embodiments, M has structure of

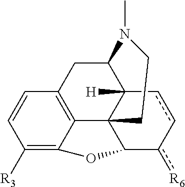

wherein R₃ is OH and R₆ is O or N that is bonded with L; or
   R₆ is OH and R₃ is O that is bonded with L.

In certain embodiments, M has structure of

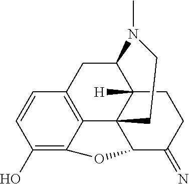 [SMILES: CN1CC[C@]23c4c5ccc(O)c4O[C@H]2C(=N)CC[C@H]3[C@H]1C5]

wherein the imine N atom is bonded with L.

In certain embodiments, M has structure of

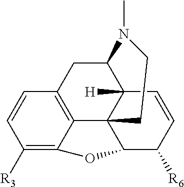

wherein R₃ is OH, and R₆ is O that is bonded with L; or
   R₆ is OH, and R₃ is O that is bonded with L.

In certain embodiments, the linker L has a molecular weight of from about 20 daltons to about 20,000 daltons. In certain embodiments, the linker has a molecular weight of from about 20 daltons to about 10,000 daltons. In certain embodiments, the linker has a molecular weight of from about 20 daltons to about 5,000 daltons. In certain embodiments, the linker has a molecular weight of from about 20 daltons to about 3,000 daltons. In certain embodiments, the linker has a molecular weight of from about 20 daltons to about 2,000 daltons. In certain embodiments, the linker has a molecular weight of from about 20 daltons to about 1,000 daltons.

In certain embodiments, the linker L is a divalent, branched or unbranched, saturated or unsaturated, hydrocarbon chain having from 2 to 30 carbon atoms, wherein one or more of the carbon atoms is optionally replaced by a divalent heterocycle (e.g., pyrrolidinyl), (—O—), (—S—), or —N(R_{b})—, wherein R_{b} is H or (C₁-C₆)alkyl, wherein the hydrocarbon chain is optionally substituted on carbon with one or more substituents selected from the group consisting of halo, hydroxy, and oxo(═O), wherein the divalent heterocycle is optionally substituted with one or more substituents selected from the group consisting of halo, hydroxy, and oxo(═O).

In certain embodiments, the linker L is a divalent, branched or unbranched, saturated or unsaturated, hydrocarbon chain having from 2 to 30 carbon atoms, wherein one or more of the carbon atoms is optionally replaced by (—O—), (—S—), —N(R_{b})—, wherein R_{b} is H or (C1-C₆)alkyl, wherein the hydrocarbon chain is optionally substituted on carbon with one or more substituents selected from the group consisting of halo, hydroxy, and oxo(═O).

In certain embodiments, the linker L is capable of forming an amide bond with the carrier protein.

In certain embodiments, the linker L is capable of forming an amide bond (—C═(O)—NH—) with an amine group (e.g., on the side chain of a lysine) of the carrier protein.

In certain embodiments, the linker L is capable of forming an amide bond (—NH—C═(O)—) with a carboxy group (e.g., on the side chain glutamic acid or aspartic acid) of the carrier protein.

In certain embodiments, the conjugate has structure of Formula Ia:

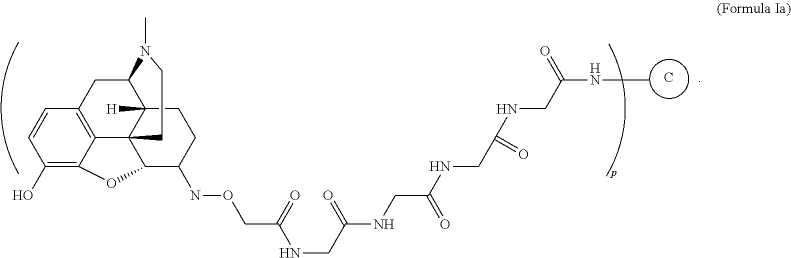

In certain embodiments, the conjugate has structure of Formula Ib:

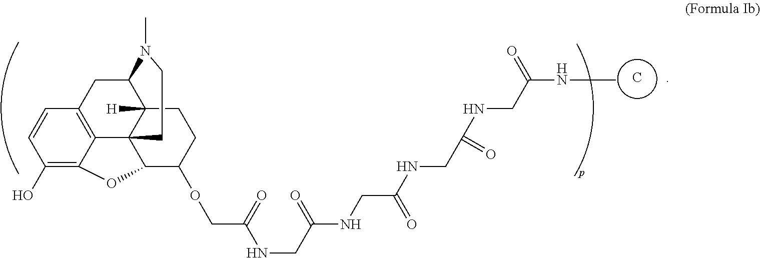

In certain embodiments, a conjugate described herein (e.g., Formula Ib) is made by using a morphine hapten described herein (e.g., see Example 10) or made by a method described herein (e.g., see Example 10, and Example 4).

As used herein, “hapten” refers to a small molecule compound (MW no greater than 1000 g/mol) that, when conjugated to a carrier protein, could elicit a specific immune response or specific antibodies against the hapten. In certain embodiments, the hapten is a drug (e.g., an opioid compound, such as morphine) or derivative thereof. A drug derivative may have structural modification on the drug, for example, a drug derivative may comprise the drug moiety, and a functional group that may be a linker, or a portion of a linker.

As used herein, “carrier protein” refers to an immunogenic protein that is exogenous to a subject (e.g., a mammal) so that administration of the carrier protein to the subject may elicit an immune response against the carrier protein and/or a hapten conjugated to the carrier protein. Carrier proteins suitable for hapten conjugation are known in the art and described herein.

In certain embodiments, the carrier protein is Keyhole limpet hemocyanin (KLH), for example, native KLH (nKLH) or subunit KLH (sKLH).

In certain embodiments, the carrier protein is native KLH (nKLH). Native KLH may be in the form of didecamer (MW about 8 MDa), and/or multidecamer (MW about 12-32 MDa).

In certain embodiments, the carrier protein is subunit KLH (sKLH). Subunit KLH (sKLH) may comprise KLH1 and/or KHL2 subunit(s). Subunit KLH (sKLH) may have MW of about 350 kDa to 400 kDa.

In certain embodiments, the carrier protein is cross-reacting material (CRM, which is a non-toxic version of diphtheria toxin), tetanus toxoid (TT), meningococcal outer membrane protein complex (OMPC), diphtheria toxoid (DT), or H. influenzae protein D (HiD).

In certain embodiments, the linker comprises a peptide segment. In certain embodiments, the linker comprises a peptide segment comprising 2, 3, 4, 5, or 6 amino acid residues. In certain embodiments, the linker comprises a peptide segment of -Gly-Gly-Gly-Gly-, also referred to as (Gly)₄.

In certain embodiments, the linker comprises a —O—CH₂—C(═O)— segment.

In certain embodiments, a Morphine-tetraglycine linker hapten has structure of M-O—CH₂—C(═O)-(Gly)₄.

In certain embodiments, a Morphine-tetraglycine linker hapten has structure of

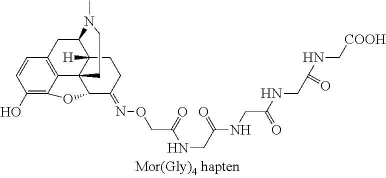 [SMILES: CN1CCC[C@]23c4c5ccc(O)c4O[C@H]2/C(=N/OCC(=O)NCC(=O)NCC(=O)NCC(=O)NCC(=O)O)CC[C@H]3[C@H]1C5]

or a salt thereof.

In certain embodiments, a Morphine-tetraglycine linker hapten has structure of

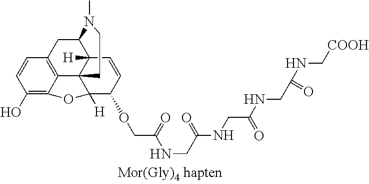

or a salt thereof.

In certain embodiments, a morphine hapten or salt thereof (such as Mor(Gly)₄ hapten) is made by a method or through an intermediate compound as described herein (e.g., see Example 10).

In certain embodiments, the conjugate has structure of

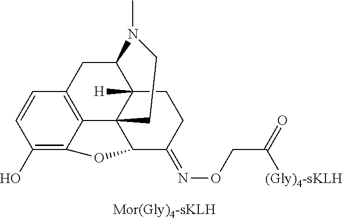

In certain embodiments, the conjugate has structure of

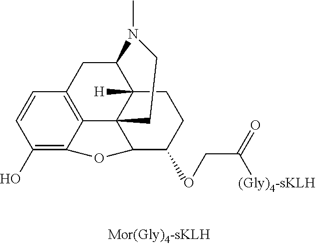

In certain embodiments, a conjugate described herein (such as Mor(Gly)₄-sKLH) is made by using a hapten described herein (e.g., see Example 10) or made by a method described herein (e.g., see Example 10 and Example 4).

Conjugation methods suitable for generating a conjugate described herein are known in the art and described herein. In certain embodiments, conjugation methods may utilize carbodiimide chemistry (e.g., see Example 4) to yield an amide bond (e.g., see Formula Ia or Formula Ib) to achieve conjugation. In certain embodiments, conjugation methods may utilize maleimide-thiol chemistry to yield a thioester bond to achieve conjugation.

In certain embodiments, the linker comprises a segment of

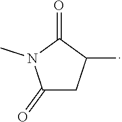

In certain embodiments, the linker comprises a segment of

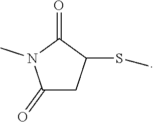

In certain embodiments, a conjugate described herein has a diameter of about 60-100 nm 65-95 nm, 70-90 nm, or 75-85 nm as determined by dynamic light scattering (DLS). In certain embodiments, a conjugate described herein has a diameter of about 60, 65, 70, 75, 80, 85, 90, or 95 nm.

In certain embodiments, a conjugate described herein has a diameter of about 150-250 nm, 160-240 nm, 170-230 nm, 180-220 nm, or 190-210 nm as determined by dynamic light scattering (DLS). In certain embodiments, a conjugate described herein has a diameter of about 150, 160, 170, 180, 190, 200, 210, 220, 230, or 240 nm.

### Synthetic Methods

Certain embodiments of the invention provide a method of preparing an intermediate compound described herein, or a morphine hapten compound or salt thereof, or a conjugate described herein. As described further in Scheme 2 of Example 10, para-methoxybenzyl (PMB) could serve as a selective protecting group for 3-hydroxyl of morphine to provide high yield for intermediate product compound 2 (from step 1 in Scheme 2) and to provide stable alkylation intermediate product compound 3 (from step 2 in Scheme 2).

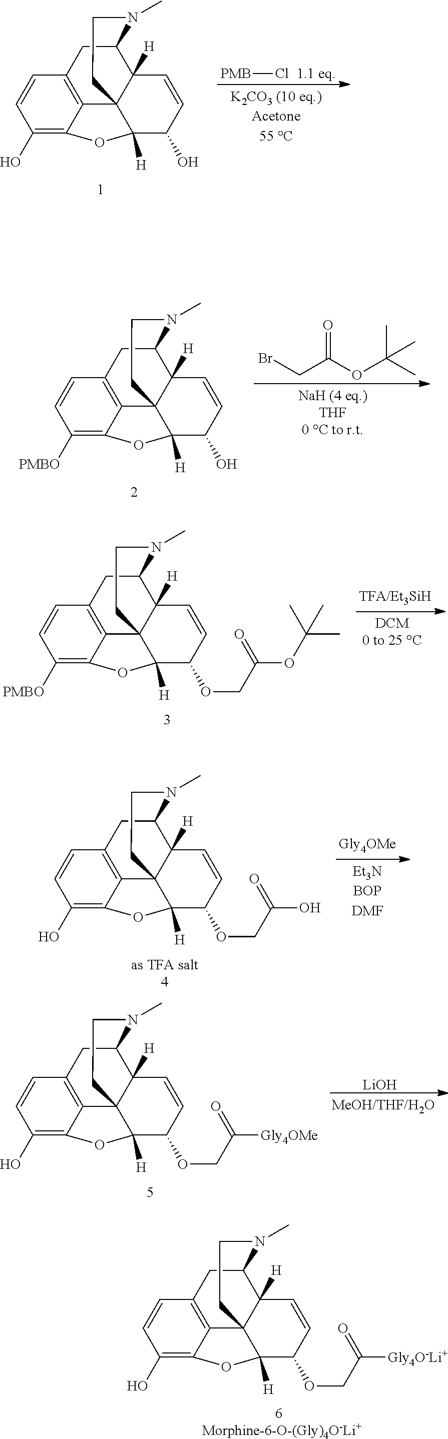

An Exemplary Synthetic route used to produce Morphine-6-O-(Gly)₄O⁻Li⁺

Accordingly, certain embodiments of the invention provide a method (e.g., a method of preparing compound 2, compound 3, compound 4, compound 5, or compound 6, or a conjugate of Formula I, such as Formula Ib), comprising converting morphine (compound 1) to (4R,4aR,7S,7aR,12bS)-9-((4-methoxybenzyl)oxy)-3-methyl-2,3,4,4a,7,7a-hexahydro-1H-4,12-methanobenzofuro[3,2-e]isoquinolin-7-ol (compound 2).

In certain embodiments, the converting comprises reacting compound 1 with para-methoxybenzyl chloride.

In certain embodiments, the method further comprises:
(b) converting compound 2 to compound 3 in an alkylation reaction (e.g., tert-butyl acetate alkylation reaction). In certain embodiments, the converting comprises reacting compound 2 with tert-butyl bromo acetate. In certain embodiments, the converting is carried out in the presence of sodium hydride (NaH), such as contacting compound 2 with NaH and tert-butyl bromo acetate.

In certain embodiments, the method further comprises:
(c) converting compound 3 to compound 4 in a deprotection reaction (e.g., contacting compound 3 with trifluoracetic acid (TFA) in the presence of triethyl silane (TES), or contacting compound 3 with trifluoracetic acid (TFA) in the presence of thioanisole).

In certain embodiments, the method further comprises:
(d) converting compound 4 to compound 5 (e.g., contacting compound 4 with Gly₄OMe in the presence of benzotriazol-1-yloxytris(dimethylamino)phosphonium hexafluorophosphate (BOP)).

In certain embodiments, the method further comprises:
(e) converting compound 5 to compound 6.

In certain embodiments, the method further comprises:
(f) contacting compound 6 with a carrier protein (e.g., sKLH, also see Example 4) in a conjugation reaction (e.g., in the presence of EDAC).

### Vaccine Compositions

Certain embodiments provide a composition described herein (e.g., in Examples 1-7).

In certain embodiments, the vaccine composition comprises one or more adjuvant, for example, in certain embodiments, the vaccine composition comprises aluminum.

In certain embodiments, the vaccine composition comprises a TLR7/8 agonist. In certain embodiments, the TLR7/8 agonist is an oxoadenine compound that may be modified with phospholipid (e.g., PEGylated di-palmitoyl glycerol). In certain embodiments, the TLR7/8 agonist is an imidazoquinoline compound (e.g., R848 (Resiquimod)).

In certain embodiments, the TLR7/8 agonist is INI-4001.

In certain embodiments, the TLR7/8 agonist has structure of Formula II:

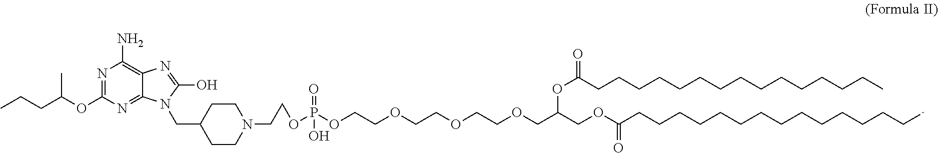

In certain embodiments, the TLR7/8 agonist has structure:

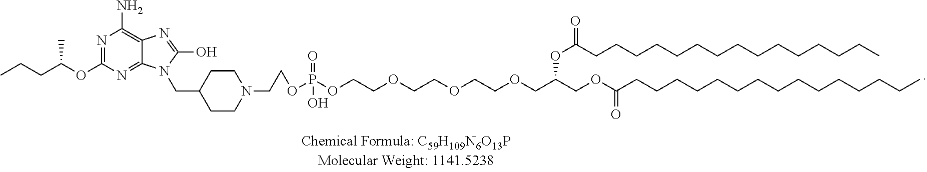 [SMILES: CCCCCCCCCCCCCCCC(=O)OC[C@H](COCCOCCOCCOP(=O)(O)OCCN1CCC(Cn2c(O)nc3c(N)nc(O[C@@H](C)CCC)nc32)CC1)OC(=O)CCCCCCCCCCCCCCC]

In certain embodiments, the TLR7/8 agonist is 6-Amino-2-[(1-S)-1-methylbutyloxy]-9-[2-(1,2-dipalmitoyl-sn-glycero-3-triethyleneglycol-phospho)ethyl-1-(4-methylenpiperidinyl]-7,9-dihydro-8H-purin-8-one.

As used herein, the term “adjuvant” refers to substance or compound that could enhance immune response against an immunogen or antigen (e.g., including hapten-carrier protein conjugate). Exemplary adjuvants include, but not limited to, Complete Freund's Adjuvant (CFA), Incomplete Freund's Adjuvant (IFA), squalene, ASO4, aluminum salt (e.g., aluminum hydroxide, aluminum phosphate, alum (e.g., potassium aluminum sulfate), or mixed aluminum salts), and toll like receptor agonist (e.g., monophosphoryl lipid A (MPL-A), a lipopeptide, and a synthetic nucleic acid sequence such as CpG and poly(I:C)).

In certain embodiments, the vaccine composition does not comprise a morphine-carrier protein conjugate having structure of Formula I, wherein M has structure of

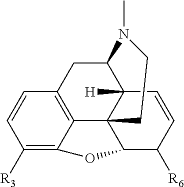

and wherein R₆ is OH and R₃ is O that is bonded with L.

In certain embodiments, the morphine-carrier protein conjugate comprised within the vaccine composition consists of a morphine-carrier protein conjugate having structure of Formula I, wherein M has structure of

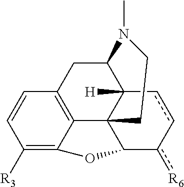

and wherein R₃ is OH and R₆ is O or N that is bonded with L (e.g., see Formula Ia or Ib).

In certain embodiments, the morphine-carrier protein conjugate comprised within the vaccine composition consists of a morphine-carrier protein conjugate having structure of Formula I, wherein M has structure of

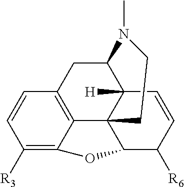

and wherein R₃ is OH and R₆ is O that is bonded with L (e.g., see Formula Ib).

In certain embodiments, the vaccine composition comprises morphine-carrier protein conjugate and aluminum at a weight ratio of about 2:3, 1:1, 1:2, 1:3, 1:4, or 1:5. In certain embodiments, the vaccine composition comprises morphine-carrier protein and aluminum at a weight ratio of about 2:3, or 1:5. In certain embodiments, the vaccine composition comprises morphine-carrier protein and aluminum at a weight ratio of about 1:1 to 1:5. In certain embodiments, the vaccine composition comprises morphine-carrier protein and aluminum at a weight ratio of about 1:1 to 1:2. In certain embodiments, the vaccine composition comprises morphine-carrier protein and aluminum at a weight ratio of about 2:3 to 1:5. In certain embodiments, the vaccine composition comprises morphine-carrier protein and aluminum at a weight ratio of about 2:3 to 1:3. In certain embodiments, the vaccine composition comprises morphine-carrier protein and aluminum at a weight ratio of about 2:3 to 1:2. In certain embodiments, the vaccine composition comprises morphine-carrier protein and aluminum at a weight ratio of about 2:3.

In certain embodiments, the vaccine composition comprises morphine-carrier protein and INI-4001 at a weight ratio of about 1:2, 3:5, 1:1, 2:1, 3:1, 4:1, 5:1, or 6:1. In certain embodiments, the vaccine composition comprises morphine-carrier protein and INI-4001 at a weight ratio of about 1:2, 3:5, or 6:1. In certain embodiments, the vaccine composition comprises morphine-carrier protein and INI-4001 at a weight ratio of about 1:2 to 6:1. In certain embodiments, the vaccine composition comprises morphine-carrier protein and INI-4001 at a weight ratio of about 4:1 to 6:1. In certain embodiments, the vaccine composition comprises morphine-carrier protein and INI-4001 at a weight ratio of about 5:1 to 6:1 (e.g., about 5.2:1 to 5.6:1, such as about 5.3:1).

In certain embodiments, the vaccine composition comprises morphine-carrier protein conjugate, aluminum, and INI-4001 that are at a weight ratio of about 6:9:1, or 5.3:8:1, or 1:5:2.

In certain embodiments, the vaccine composition comprises about 10-800 μg morphine-carrier protein conjugate. In certain embodiments, the vaccine composition comprises about 20-600 μg morphine-carrier protein conjugate. In certain embodiments, the vaccine composition comprises about 25-400 μg morphine-carrier protein conjugate. In certain embodiments, the vaccine composition comprises about 5, 10, 20, 30, 40, 50, 60, 70, 80, 90, 100, 150, 200, 250, 300, 350, 400, 450, 500, 550, 600, 650 700, 750, or 800 μg morphine-carrier protein conjugate. In certain embodiments, the vaccine composition comprises about 40, 60, 100, or 400 μg morphine-carrier protein conjugate.

In certain embodiments, the vaccine composition comprises about 15-1200 μg aluminum. In certain embodiments, the vaccine composition comprises about 30-900 μg aluminum. In certain embodiments, the vaccine composition comprises about 35-600 μg aluminum. In certain embodiments, the vaccine composition comprises about 10, 20, 30, 40, 50, 60, 70, 80, 90, 100, 150, 200, 250, 300, 350, 400, 450, 500, 550, 600, 650 700, 750, 800, 850, 900, 950, 1000, 1100, or 1200 μg aluminum. In certain embodiments, the vaccine composition comprises about 60, 90, 150, or 600 μg aluminum.

In certain embodiments, the vaccine composition comprises about 1.8-150 μg INI-4001. In certain embodiments, the vaccine composition comprises about 3.8-115 μg INI-4001. In certain embodiments, the vaccine composition comprises about 4.7-75 μg INI-4001. In certain embodiments, the vaccine composition comprises about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 μg INI-4001. In certain embodiments, the vaccine composition comprises about 7.5-75 μg INI-4001. In certain embodiments, the vaccine composition comprises about 7.5, 18.8, or 75 μg INI-4001.

In certain embodiments, the vaccine composition comprises a morphine-sKLH carrier protein conjugate, and INI-4001, as described herein.

In certain embodiments, the vaccine composition comprises a morphine-sKLH carrier protein conjugate, aluminum, and INI-4001, as described herein.

In certain embodiments, the vaccine composition further comprises a surfactant (e.g., polysorbate 80 such as 0.1% or 0.01% PS80). In certain embodiments, the vaccine composition comprises about 0.01%-0.1% (w/v) polysorbate 80. In certain embodiments, the vaccine composition comprises about 0.01%-0.09%, 0.01%-0.08%, 0.01%-0.07%, 0.01%-0.06%, 0.01%-0.05%, 0.01%-0.04%, 0.01%-0.03% or 0.01%-0.02% polysorbate 80. In certain embodiments, the vaccine composition comprises about 0.01% (0.1 mg/mL), 0.02% (0.2 mg/mL), 0.03%, 0.04%, 0.05%, 0.06%, 0.07%, 0.08%,or 0.09% polysorbate 80.

In certain embodiments, the vaccine composition further comprises an antioxidant. In certain embodiments, the antioxidant is monothioglycerol (MTG). In certain embodiments, the vaccine composition comprises about 0.1% to 0.9% (w/v), 0.2% to 0.8%, 0.3% to 0.7%, or 0.4% to 0.6% antioxidant (e.g., MTG). In certain embodiments, the vaccine composition comprises about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, or 0.9% antioxidant (e.g., MTG).

### Methods

Certain embodiments of the invention provide a method of treating opioid use disorder (OUD) or immunizing a subject (e.g., for the treatment or prevention of overdose), comprising administering a vaccine composition described herein to the subject.

Certain embodiments of the invention provide a method of protecting a subject from opioid overdose or overdose toxicity, comprising administering a vaccine composition described herein to the subject.

In certain embodiments, the OUD is Heroin use disorder (HUD).

In certain embodiments, the method comprises administering a vaccine composition described herein to the subject, wherein morphine specific antibody, heroin specific antibody, and/or 6-AM specific antibody is induced in the subject.

In certain embodiments, the subject is a mammal (e.g., human, mouse, rat, hamster, monkey, mini-pig, rabbit, goat, pig, llama, camel, or horse).

In certain embodiments, the subject is protected against heroin self-administration. In certain embodiments, a method described herein reduces opioid (e.g., heroin) drug seeking behavior of the subject. In certain embodiments, a method described herein reduces the subject's opioid (e.g., heroin) drug dependence.

In certain embodiments, the subject is protected against heroin-induced respiratory depression or cardiovascular toxicity. In certain embodiments, a method described herein reduces opioid (e.g., heroin) depressant effects in the subject. In certain embodiments, a method described herein reduces the subject's opioid (e.g., heroin) toxicity associated with fatal and non-fatal overdose.

In certain embodiments, the method further comprises administering to the subject an additional therapeutic agent (e.g., overdose reversal agent, such as naloxone).In certain embodiments, the method further comprises administering to the subject an additional OUD therapeutic agent. In certain embodiments, the additional OUD therapeutic agent is methadone. In certain embodiments, the additional OUD therapeutic agent is buprenorphine.

In certain embodiments, the subject is immunized with 1 dose of the vaccine composition. In certain embodiments, the subject is immunized with one or more additional dose (e.g., 1, 2, 3, or more booster dose) of the vaccine composition. In certain embodiments, the subject is immunized with 1, 2, 3, 4 or more doses of the vaccine composition. A booster dose may be given 2, 3, 4, 5, 6, or more weeks after the original or a previous booster dose was administered. In certain embodiments, a sequence of booster doses (e.g., 2 or 3 booster doses) are administered wherein individual dose is administered every 2, 3, 4, 5 or 6 weeks.

In certain embodiments, the method or vaccine composition described herein induces or is capable of inducing in the subject a heroin, 6-MAM, and/or morphine-specific antibody titer that is at least 15% (e.g., 20%, 30%, 40%, 50%, 60%, 70%, 80%, 90%, 100%, 150%, 200% or more) higher as compared to a control (e.g., vehicle control, or same vaccine without TLR7/8 agonist, such as INI-4001). In certain embodiments, the method or vaccine composition described herein induces or is capable of inducing in the subject a heroin, 6-MAM, and/or morphine-specific antibody titer that is at least 50% (e.g., 60%, 70%, 80%, 90%, 100%, or more) higher as compared to a control (e.g., vehicle control, or same vaccine without TLR7/8 agonist, such as INI-4001).

In certain embodiments, the method or vaccine composition described herein induces or is capable of inducing in the subject a 6-AM-specific antibody titer that is at least 15% (e.g., 20%, 30%, 40%, 50%, 60%, 70%, 80%, 90%, 100%, 150%, 200% or more) higher as compared to a control (e.g., vehicle control, or same vaccine without TLR7/8 agonist, such as INI-4001).

In certain embodiments, the method or vaccine composition described herein protects or is capable of protecting the subject against heroin-induced respiratory depression (e.g., as measured by % reduction of oxygen saturation). In certain embodiments, the protected subject maintains above 80%, 81%, 82%, 83%, 84%, 85%, 86%, 87%, 88%, 89%, 90% oxygen saturation level despite being under heroin influence.

In certain embodiments, the method or vaccine composition described herein does not interfere with mu-receptor antagonist (e.g., naloxone) based pharmacotherapy.

In certain embodiments, the method or vaccine composition described herein protects or is capable of protecting the subject against heroin-induced bradycardia.

In certain embodiments, the method or vaccine composition described herein induces or is capable of inducing in the subject heroin-specific antibody.

In certain embodiments, the method or vaccine composition described herein induces or is capable of inducing in the subject heroin-specific antibody titer that is at least 15% (e.g., 20%, 30%, 40%, 50%, 60%, 70%, 80%, 90%, 100%, 150%, 200% or more) higher as compared to a control (e.g., not immunized, receiving vehicle control, or receiving same vaccine without TLR7/8 agonist, such as INI-4001).

In certain embodiments, the method or vaccine composition described herein sequesters or is capable of sequestering heroin in the blood (e.g., as measured by serum heroin concentration after heroin exposure or challenge). In certain embodiments, the method or vaccine composition described herein sequesters or is capable of sequestering heroin metabolite(s) (6-acetylmorphine and/or morphine) in the blood (e.g., as measured by serum heroin metabolite(s) (6-acetylmorphine and/or morphine) concentration after heroin exposure or challenge).

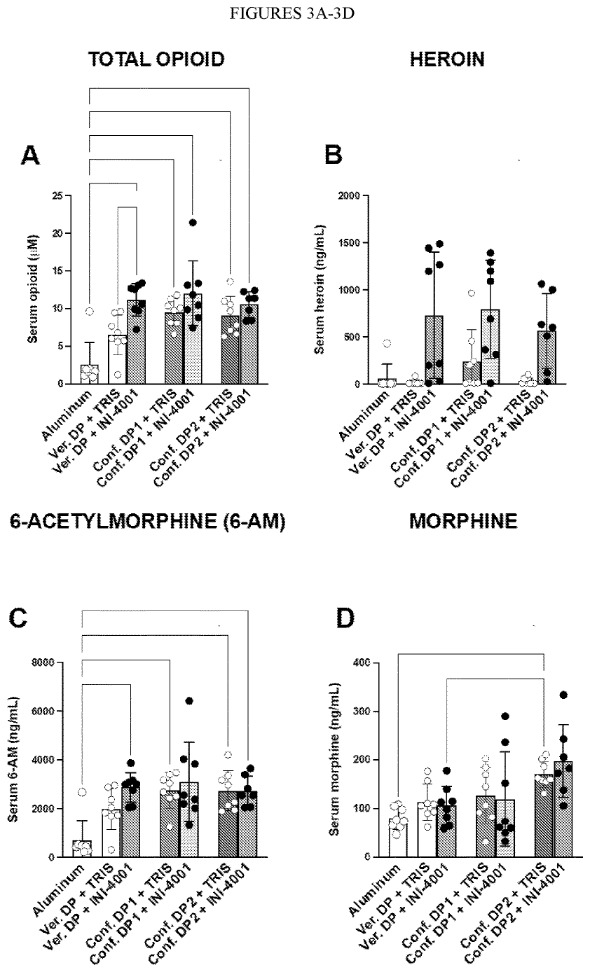

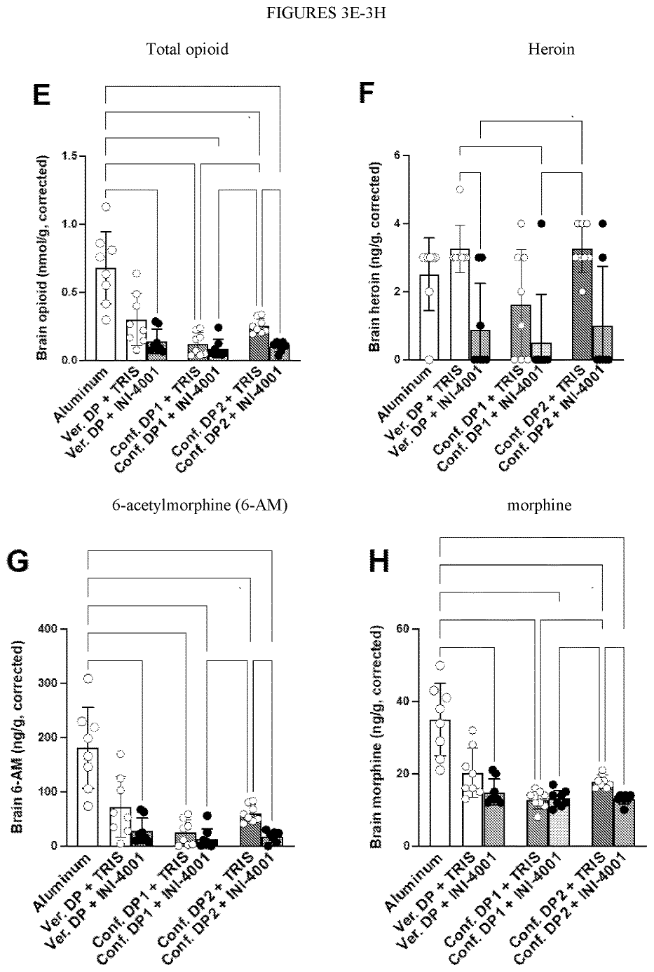

In certain embodiments, the method or vaccine composition described herein sequesters or is capable of sequestering heroin in the blood of the subject who has a serum heroin concentration that is at least 15% (e.g., 20%, 30%, 40%, 50%, 60%, 70%, 80%, 90%, 100%, 150%, 200% or more) higher as compared to a control (e.g., not immunized, receiving vehicle control, or receiving same vaccine without TLR7/8 agonist, such as INI-4001). It is also unexpectedly found herein that the method or vaccine composition having TLR7/8 agonist, such as INI-4001, as described herein may preferentially or selectively enhance heroin serum sequestration over 6-AM and/or morphine serum sequestration as compared to a control (e.g., receiving same vaccine without TLR7/8 agonist such as INI-4001) (also see, e.g., FIGS. 3B-3D that shows heroin serum sequestration is significantly enhanced by TLR7/8 agonist, such as INI-4001, while the effects on 6-AM and/or morphine serum sequestration is not as significant).

In certain embodiments, the method or vaccine composition described herein reduces or is capable of reducing heroin in the brain (e.g., as measured by brain heroin concentration after heroin exposure or challenge).

In certain embodiments, the method or vaccine composition described herein reduces or is capable of reducing heroin in the brain of the subject who has a brain heroin concentration that is at least 15% (e.g., 20%, 30%, 40%, 50%, 60%, 70%, 80%, 90%, 100%, 150%, 200% or more) lower as compared to a control (e.g., not immunized, receiving vehicle control, or receiving same vaccine without TLR7/8 agonist, such as INI-4001).

In certain embodiments, the method or vaccine composition described herein reduces or is capable of reducing total opioid, 6-AM, or morphine in the brain of the subject who has a brain concentration of total opioid, 6-AM, or morphine that is at least 15% (e.g., 20%, 30%, 40%, 50%, 60%, 70%, 80%, 90%, 100%, 150%, 200% or more) lower as compared to a control (e.g., not immunized, receiving vehicle control, or receiving same vaccine without TLR7/8 agonist, such as INI-4001).

In certain embodiments, the method or vaccine composition described herein reduces or is capable of reducing heroin-induced antinociception (e.g., as measured by antinociception EC50 or latency to respond) in the subject by at least 15% (e.g., 20%, 30%, 40%, 50%, 60%, 70%, 80%, 90%, 100%, 150%, 200% or more) as compared to a control (e.g., not immunized, receiving vehicle control, or receiving same vaccine without TLR7/8 agonist, such as INI-4001).

In certain embodiments, the method or vaccine composition described herein sequesters or is capable of sequestering 6-AM in the blood of the subject who has a serum 6-AM concentration that is at least 15% (e.g., 20%, 30%, 40%, 50%, 60%, 70%, 80%, 90%, 100%, 150%, 200% or more) higher as compared to a control (e.g., not immunized, receiving vehicle control, or receiving same vaccine without TLR7/8 agonist, such as INI-4001).

In certain embodiments, the method or vaccine composition described herein sequesters or is capable of sequestering morphine in the blood of the subject who has a morphine concentration that is at least 15% (e.g., 20%, 30%, 40%, 50%, 60%, 70%, 80%, 90%, 100%, 150%, 200% or more) higher as compared to a control (e.g., not immunized, receiving vehicle control, or receiving same vaccine without TLR7/8 agonist, such as INI-4001).

In certain embodiments, the method or vaccine composition described herein protects or is capable of protecting the subject against heroin-induced bradycardia (e.g., as measured by heart rate). In certain embodiments, the protected subject maintains above about 60%, 65%, 70%, 71%, 72%, 73%, 74%, 75%, 76%, 77%, 78%, 79%, 80% of original heart rate despite being under heroin influence, as compared to a control (e.g., not immunized, receiving vehicle control, or receiving same vaccine without TLR7/8 agonist, such as INI-4001) that fails to do so.

Methods of testing antibody titer or opioid compound(s) tissue or serum concentration are known in the art and described herein (e.g., see Examples). In certain embodiments, the antibody titer, or s opioid compound(s) concentration, is tested at about 1 week, 2 weeks, 3 weeks, 1 month, 2 months, or 3 months after the completion of the vaccination regimen (e.g., 1-3 weeks after the last booster shot).

### Administration

In certain embodiments, an effective amount of a conjugate, or composition described herein is administered to the subject. “Effective amount” or “therapeutically effective amount” or “immunologically effective amount” are used interchangeably herein, and refer to an amount of a conjugate, or composition, as described herein effective to achieve a particular biological result.

In certain embodiments, the vaccine composition is a liquid composition.

In certain embodiments, the composition is a solid composition (e.g., lyophilized) that may be reconstituted with suitable solvent (e.g., saline or Dextrose 5% in water) prior to administration.

In certain embodiments, a conjugate, or composition described herein is administered via intramuscular, intradermal, or subcutaneous delivery.

As used herein, “immunization” or “vaccination” are used interchangeably herein and are intended for prophylactic or therapeutic immunization or vaccination.

As used herein, “pharmaceutically acceptable” refers to those properties and/or substances which are acceptable to the subject or patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium (e.g., aqueous medium) that does not interfere with the effectiveness of the biological activity of the active ingredient(s) and is not toxic to the host to which it is administered.

In certain embodiments, a composition described herein comprises a pharmaceutically acceptable carrier (e.g., aqueous medium).

The conjugate, or composition of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present conjugate, or composition may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the conjugate, or composition may be combined with one or more excipients and used in the form of ingestible tablets, buccal tablets, troches, capsules, elixirs, suspensions, syrups, wafers, and the like. Such compositions and preparations should contain at least 0.1% of conjugate. The percentage of the compositions and preparations may, of course, be varied and may conveniently be between about 2 to about 60% of the weight of a given unit dosage form. The amount of the conjugate in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the conjugate, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the conjugate or vaccine composition may be incorporated into sustained-release preparations and devices.

The conjugate, or composition may also be administered intravenously or intraperitoneally by infusion or injection. Solutions of the conjugate can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the conjugate which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the conjugate in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the conjugate plus any additional desired ingredient present in the previously sterile-filtered solutions.

For topical administration, the present conjugate, or composition may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present conjugate can be dissolved or dispersed at effective levels, optionally with the aid of non-toxic surfactants. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the conjugate, or composition to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

To immunize a subject, a conjugate, or composition described herein is administered parenterally, usually by intramuscular or subcutaneous injection in an appropriate vehicle. Other modes of administration, however, such as oral delivery or intranasal delivery, are also acceptable. Vaccine formulations will contain an effective amount of the active ingredient (conjugate) in a vehicle.

Formulations will contain an effective amount of the active ingredient in a vehicle. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal considered for vaccination and kind of concurrent treatment, if any. The quantity also depends upon the capacity of the animal's immune system to synthesize antibodies, and the degree of protection desired. Typically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference. Additionally, effective dosages can be established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

Useful dosages of the compositions of the present invention can be determined by comparing their in vitro activity, and in vivo activity in animal models. The amount of the compositions described herein required for use in treatment will vary with the route of administration and the age and condition of the subject and will be ultimately at the discretion of the attendant veterinarian or clinician.

Certain embodiments of the invention are described herein, for example,
Embodiment 1. A conjugate having structure of Formula I.

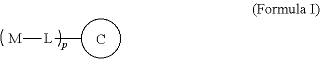

wherein M is morphine, L is a linker, C is a carrier protein, and
p is a hapten to carrier protein ratio number that is >=1 (e.g., 1-50, 1-30, or 1-10).
Embodiment 2. The conjugate of Embodiment 1, wherein M has structure of

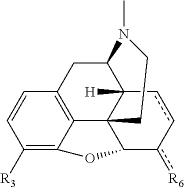

wherein R₃ is OH and R₆ is O or N that is bonded with L; or
   R₆ is OH and R₃ is O that is bonded with L.
Embodiment 3. The conjugate of Embodiment 1, wherein M is morphine having structure of

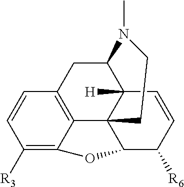

wherein R₃ is OH, and R₆ is O that is bonded with L; or
   R₆ is OH, and R₃ is O that is bonded with L.
Embodiment 4. The conjugate of Embodiment 1, wherein M is morphine having structure of

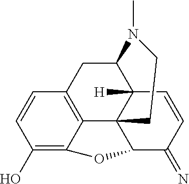 [SMILES: CN1CC[C@]23c4c5ccc(O)c4O[C@H]2C(=N)C=C[C@H]3[C@H]1C5]

wherein the imine N atom is bonded with L.
Embodiment 5. The conjugate of any one of Embodiments 1-4, wherein C is Keyhole limpet hemocyanin (e.g., native KLH or subunit KLH).
Embodiment 6. The conjugate of Embodiment 5, wherein C is subunit KLH (sKLH).
Embodiment 7. The conjugate of Embodiment 2, wherein R₃ is OH, and R₆ is O.
Embodiment 8. The conjugate of Embodiment 2, wherein R₆ is OH, and R₃ is O.
Embodiment 9. The conjugate of any one of Embodiments 1-8, wherein L comprises a -Gly-Gly-Gly-Gly-peptide segment.
Embodiment 10. The conjugate of Embodiment 3, having structure of Formula Ia:

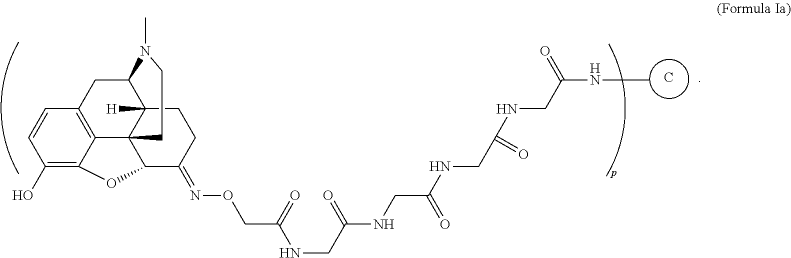

Embodiment 11. The conjugate of Embodiment 2 or 7, having structure of Formula Ib:

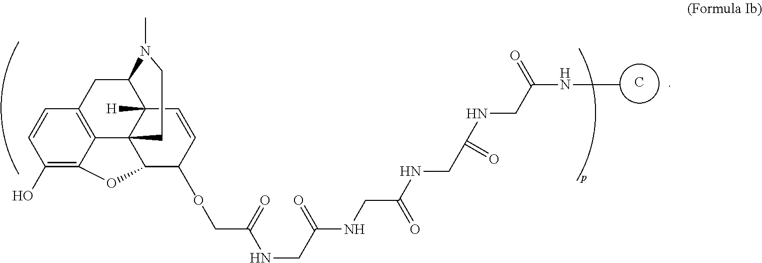

Embodiment 12. A vaccine composition comprising a conjugate of any one of Embodiments 1-11.
Embodiment 13. The vaccine composition of Embodiment 12, further comprising one or more adjuvant.
Embodiment 14. The vaccine composition of Embodiment 13, wherein the one or more adjuvant comprise aluminum.
Embodiment 15. The vaccine composition of Embodiment 13 or 14, wherein the one or more adjuvant comprise INI-4001 having structure of:

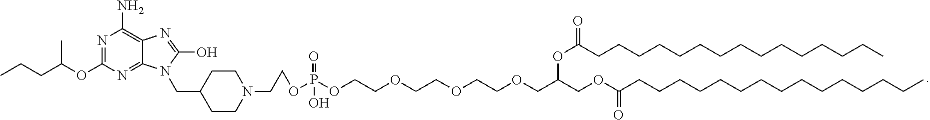 [SMILES: CCCCCCCCCCCCCCCC(=O)OCC(COCCOCCOCCOP(=O)(O)OCCN1CCC(Cn2c(O)nc3c(N)nc(OC(C)CCC)nc32)CC1)OC(=O)CCCCCCCCCCCCCCC]

Embodiment 16. A method of treating opioid use disorder (OUD) or immunizing a subject, comprising administering a vaccine composition described herein to the subject.
Embodiment 17. The method of Embodiment 16, wherein heroin specific antibody is induced in the subject.

### Certain Definitions

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a conjugate of formula I through the carboxy terminus, the amino terminus, or through any other convenient point of attachment, such as, for example, through the sulfur of a cysteine.

The terms “treat” and “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a conjugate or vaccine of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, a “subject” is an animal, e.g., a mammal, e.g., a human, monkey, dog, cat, horse, cow, pig, mini-pig, goat, rabbit, rat, hamster, or mouse.

The term “heterocycle” or “heterocycloalkyl” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” or “heterocycloalkyl” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The invention will now be illustrated by the following non-limiting Examples.

### Example 1. TLR7/8 Agonist INI-4001 Improves Efficacy of Heroin Vaccine Intended for Clinical Treatment of Opioid Use Disorder

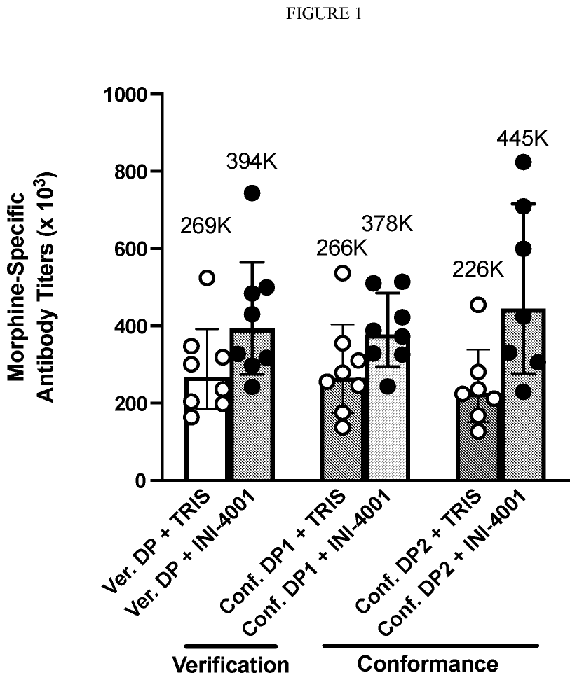

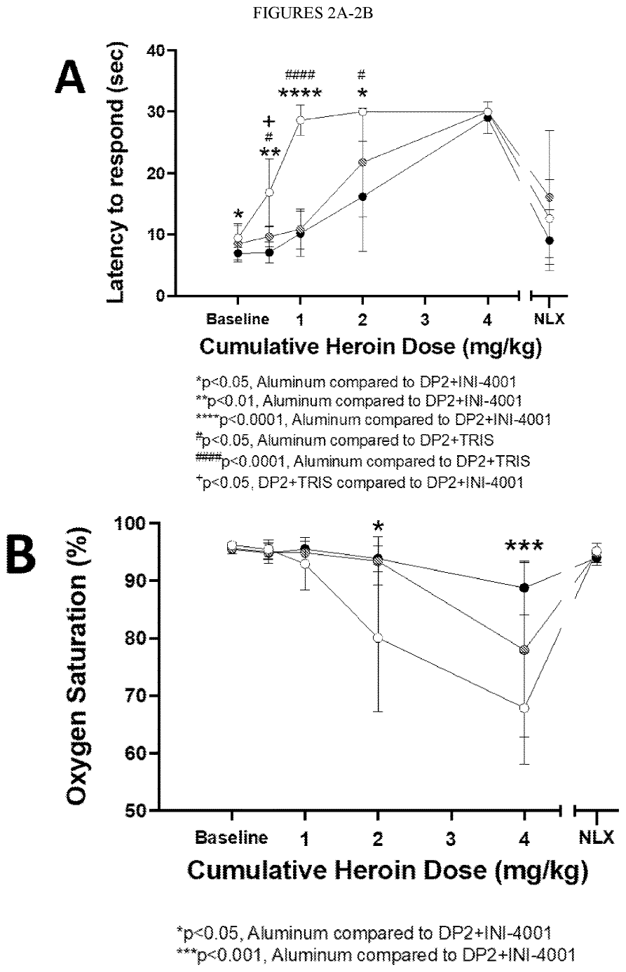

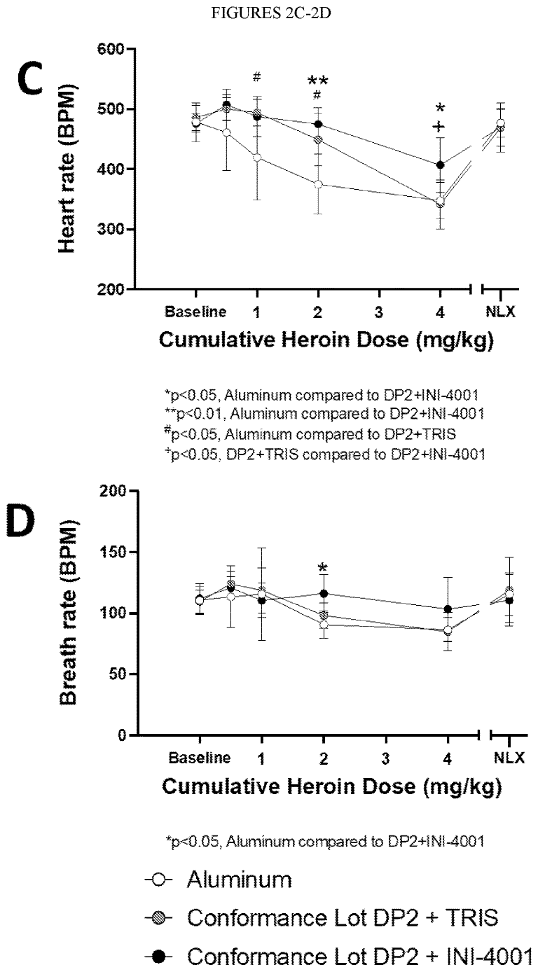

A heroin vaccine (M-sKLH) to treat opioid use disorder (OUD) is being prepared for clinical testing. Currently, a TLR7/8 agonist, INI-4001, is being studied for its ability to increase vaccine efficacy in opioid use disorder vaccines. To study its effects on increasing vaccine efficacy, INI-4001 was added to M-sKLH adsorbed to aluminum and tested in rats. Male Sprague Dawley rats were vaccinated with M-sKLH adsorbed to aluminum adjuvant with either buffer or INI-4001 and compared against controls having aluminum alone. Cumulative doses of heroin (from 0.5 up to 4 mg/kg, s.c.) were given every 15 minutes and then rats were tested for antinociception via hotplate and respiratory depression via oximetry. Following the final dosing, rats receive a dose of naloxone. Morphine-specific antibody titers were doubled in the group containing INI-4001 (FIG. 1). Both M-sKLH with aluminum and M-sKLH with aluminum and INI-4001 showed protection against heroin-induced antinociception (FIG. 2A). However, only M-sKLH with aluminum and INI-4001 was protective against heroin-induced respiratory depression (as measured by % reduction of oxygen saturation) (FIG. 2B). Naloxone reversed heroin-induced effects in all groups, demonstrating that antagonist activity is preserved in M-sKLH vaccinated animals. These data demonstrate that addition of INI-4001 improves the efficacy of M-sKLH adsorbed to aluminum and support the use of M-sKLH adsorbed to aluminum and mixed with INI-4001 for treatment of opioid use disorder in humans.

### Methods and Materials

Animals. All studies were approved by the Institutional Animal Care and Use Committee prior to initiation. Male Sprague-Dawley rats (225-250 g) were housed with a 14/10 light/dark cycle and fed ad libitum.

Drugs and vaccines. M-sKLH was manufactured at its original concentration (DP1: 0.4 mg/mL in 0.6 mg/mL aluminum) and at 2× concentration (DP2: 0.8 mg/mL in 1.2 mg/mL aluminum). Rats were immunized on days 0, 14, 28, and 42 with aluminum as a negative control, 60 μg verification batch Mor(Gly)4-sKLH+90 μg aluminum+Tris buffer or 10 g INI-4001, 60 μg DP1 conformance batch Mor(Gly)4-sKLH+90 μg aluminum+Tris buffer or g INI-4001, or 60 μg DP2 conformance batch Mor(Gly)4-sKLH+90 μg aluminum+Tris buffer or 10 μg INI-4001 in sterile physiological saline free of preservative at a concentration of 10 mg/mL. Heroin and naloxone were purchased through Sigma.

Behavioral assays. Antinociception was measured as latency to respond on a hot plate set to 54 °C, with a maximum latency of 30 sec to avoid tissue damage. Oxygen saturation was measured with a MouseOx Plus pulse oximeter.

Drug distribution analysis. After heroin challenge, rats were euthanized with CO₂, and blood and brain were collected for analysis of drug concentration by liquid chromatography-mass spectrometry (LC-MS).

### Results

Efficacy of heroin vaccine mixed with Tris buffer or INI-4001. Rats (n=8/group) were vaccinated on days 0, 14, 28, and 42. Verification lot (Ver.) was compared to Conformance lot (Conf.) with DP1 and DP2 material. DP2 is 2× concentrated vaccine for ease of mixing 1:1 with INI-4001 adjuvant. ANOVA showed a main effect, but no differences were seen between groups. INI-4001 showed a trend for increased antibody levels (FIG. 1).

INI-4001 increases the binding and selectivity of heroin vaccine-generated serum antibodies. One week following hotplate and physiological measurements from FIG. 2, rats received a single dose of 1 mg/kg heroin s.c., and blood and brain collected 5 minutes later. Serum and brain levels of total opioids (FIGS. 3A, E) in INI-4001 containing groups showed increased efficacy. Individual levels of heroin and its metabolites (6-acetylmorphine and morphine) are shown in panels FIGS. 3B, C, D, F, G, and H. A significant reduction in brain heroin levels were seen in the verification run samples containing INI-4001 compared to Tris buffer.

In particular, the experiment was performed using M-sKLH conformance batch material to compare the efficacy of a 1 g scale up batch of M-sKLH with and without low dose INI-4001. To allow a 1:1 combination of M-sKLH and INI-4001 in a final formulation, M-sKLH was manufactured at its original concentration (DP1: 0.4 mg/mL in 0.6 mg/mL aluminum) and at 2× concentration (DP2: 0.8 mg/mL in 1.2 mg/mL aluminum). Rats were immunized on days 0, 14, 28, and 42 with aluminum as a negative control, 60 μg verification batch Mor(Gly)₄-sKLH+90 μg aluminum+Tris buffer or 10 μg INI-4001, 60 μg DP1 conformance batch Mor(Gly)₄-sKLH+90 μg aluminum+Tris buffer or 10 μg INI-4001, or 60 μg DP2 conformance batch Mor(Gly)₄-sKLH+90 μg aluminum+Tris buffer or 10 μg INI-4001 (FIGS. 1 and 2).

One week after final immunization, serum was taken to analyze antibody titers. Morphine-specific serum IgG levels (FIG. 1) were compared using unpaired t tests between tris buffer and INI-4001 containing groups for each formulation and only DP2 containing INI-4001 showed a significant increase compared to tris buffer containing group (p<0.05), although titers appeared higher in all INI-4001 containing vaccine groups.

One week later (i.e., two weeks after final immunization), the DP2 groups and aluminum controls were given escalating SC doses of heroin (up to a cumulative dose of 4 mg/kg). Fifteen minutes after each dose, rats were assessed for latency to respond on the hotplate as well as for oxygen saturation and heart rate. DP2 with and without INI-4001 reduced heroin-induced antinociception at 0.5, 1.0, and 2.0 mg/kg heroin doses (FIG. 2A). While visually all groups had similar baseline latencies there was a significant reduction at baseline for DP2+INI-4001. Only DP2+INI-4001 showed significant protection against heroin-induced respiratory depression compared to aluminum controls (FIG. 2B), while both DP2 and DP2+INI-4001 showed protection against heroin-induced bradycardia (FIG. 2C).

One week after heroin challenge, all groups received a 1 mg/kg SC dose of heroin and serum and brain were collected 5 minutes later (FIG. 3). Serum levels (FIGS. 3A-3D) of 6-AM and morphine were increased compared to controls. Interestingly, serum heroin levels were elevated in the DP2+INI-4001 groups, although these findings were not statistically significant due to the large number of groups. Post-hoc one-way ANOVA between aluminum and DP2 formulations shows that heroin is significantly increased in DP2 formulations and that DP2+INI-4001 is significantly increased in serum compared to DP2 alone (p<0.05).

Brain opioid levels (FIGS. 3E-3H) were significantly reduced in most groups compared to aluminum alone. Importantly, DP2+INI-4001 showed significant reduction of total opioid (p<0.001), 6-AM (p<0.001), and morphine (p<0.01) compared to DP2 alone. These data demonstrate that a 2× concentrated M-sKLH formulation with aluminum mixed 1:1 with INI-4001 showed greater efficacy compared to a similar formulation mixed with Tris buffer. These data confirm that a 2× formulation (diluted 1:1 prior to vaccination) is as effective as a 1× formulation and that INI-4001 improves the efficacy of M-sKLH to protect against heroin-induced effects, providing a formulation to test in a clinical study.

### REFERENCES FOR EXAMPLE 1

Baruffaldi F, Raleigh M D, King S J, Roslawski M J, Birnbaum A K, Hassler C, Carroll F I, Runyon S P, Winston S, Pentel P R, Pravetoni M. Formulation and characterization of conjugate vaccines to reduce opioid use disorders suitable for pharmaceutical manufacturing and clinical evaluation. Mol Pharm. 2019 Jun. 3; 16(6):2364-2375.
Alyson J. Smith, Yufeng Li, Hélène G. Bazin, Julien R. St-Jean, Daniel Larocque, Jay T. Evans, and Jory R. Baldridge Evaluation of novel synthetic TLR7/8 agonists as vaccine adjuvants. Vaccine. 2016 Aug. 5; 34(36): 4304-4312. doi:10.1016/j.vaccine.2016.06.080
Raleigh M D, Laudenbach M, Baruffaldi F, Peterson S J, Roslawski M J, Birnbaum A K, Carroll F I, Runyon S P, Winston S, Pentel P R, Pravetoni M. Opioid dose-and route-dependent efficacy of oxycodone and heroin vaccines in rats. Journal of Pharmacology and Experimental Therapeutics. 2018 May; 365(2):346-353
Raleigh M D, Pentel P R, LeSage M G. Pharmacokinetic correlates of the effects of a heroin vaccine on heroin self-administration in rats. PloS One. 2014 Dec. 23; 9(12):el 15696.
Raleigh M D, Pravetoni M, Harris A C, Birnbaum A K, Pentel P R. Selective effects of a morphine conjugate vaccine on heroin and metabolite distribution and heroin-induced behaviors in rats. Journal of Pharmacology and Experimental Therapeutics. 2013 February; 344:397-406.

### Example 2. Addition of a Novel Tlr7/8 Agonist Adjuvant Enhances the Efficacy of Mor(Gly)₄-Sklh

We tested whether the addition of the novel TLR7/8 agonist INI-4001 could enhance the efficacy of the Mor(Gly)₄-sKLH vaccine against heroin. INI-4001 is a novel, small molecule TLR7/8 agonist, which is an oxoadenine core compound optimized for TLR7 and TLR8 activity in human peripheral blood mononuclear cells (PBMC). The core oxoadenine has been modified with a phospholipid (PEGylated di-palmitoyl glycerol) to further improve TLR7/8 adjuvant activity and allow for adsorption to aluminum salts. INI-4001 adjuvant can be mixed with vaccine formulations containing aluminum hydroxide (to which it readily adsorbs), and delivered via, e.g., intramuscular (IM) administration.

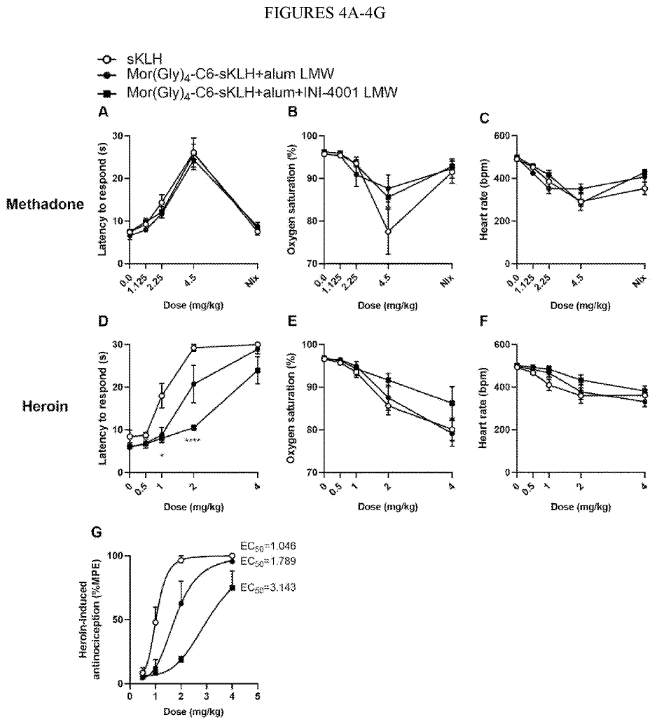

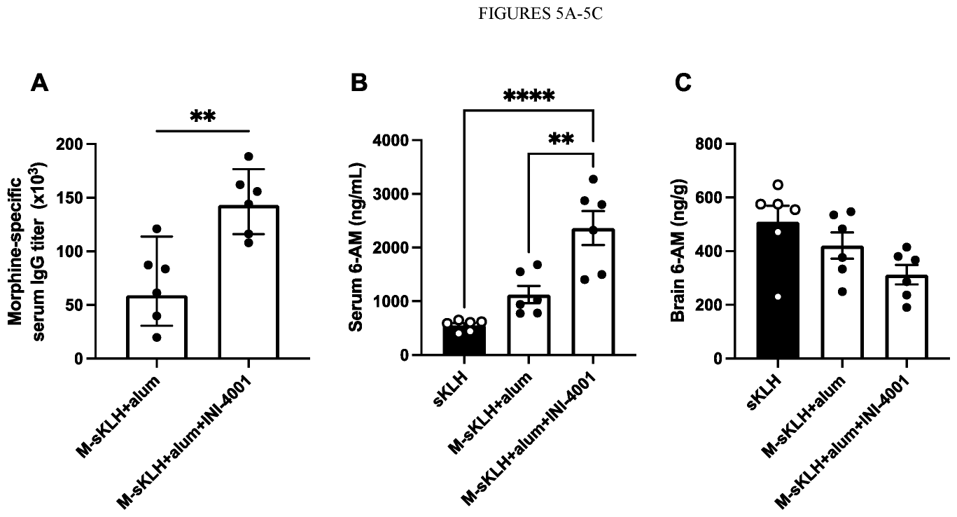

For this Example, a LMW Mor(Gly)₄-sKLH formulation was used as a representative vaccine. Rats were immunized on days 0, 21, and 42 with sKLH as a negative control, 5 g LMW Mor(Gly)₄-sKLH+24 μg aluminum, or 5 μg LMW Mor(Gly)₄-sKLH+24 μg aluminum+10 μg INI-4001. This study also included an arm with 60 μg LMW Mor(Gly)₄-sKLH+90 g aluminum+10 μg INI-4001. One week after the final immunization, titers were assessed via ELISA. The following week, rats were challenged with a cumulative dose of up to 4.5 mg/kg methadone to assess whether addition of INI-4001 changed the specificity of the polyclonal antibody response. Every 15 minutes after injection, the rats were assessed for latency to respond on a hot plate, as well as oxygen saturation and heart rate using a collar pulse oximeter before immediately receiving their next dose. After methadone challenge, there was no differences found at any dose between groups when assessing latency to respond, oxygen saturation, or heart rate (FIGS. 4A-C). Fifteen minutes after the final injection, rats were administered naloxone and latency to respond, oxygen saturation, and heart rate were measured fifteen minutes later. All groups returned to baseline in all metrics tested, suggesting that addition of INI-4001 does not interfere with naloxone reversal of opioid-induced effects.

The following week, rats were challenged with a cumulative dose of up to 4 mg/kg heroin. Every 15 minutes after injection, rats were assessed for latency to respond, oxygen saturation, and heart rate before immediately receiving their next dose. Fifteen minutes after their final dose, rats were euthanized, and blood and brain were collected for LCMS analysis. Addition of INI-4001 significantly decreased the rats' latency to respond at 1 mg/kg and 2 mg/kg cumulative heroin dose and trended toward decreased latency to respond at 4 mg/kg (FIG. 4D). Rats that received INI-4001 trended toward increased oxygen saturation at 2 mg/kg and 4 mg/kg cumulative heroin dose, although this difference was not statistically significant (FIG. 4E). When plotting the latency respond as a percent maximum possible effect (% MPE), the addition of INI-4001 increased the EC₅₀ of the dose-response curve by almost 2-fold (FIG. 4G).

Analysis of antibody levels showed that addition of INI-4001 significantly increased morphine-specific IgG titers compared to the vaccine alone (FIG. 5A). Morphine-specific serum total IgG titers were 59±1.93 (GMT±SD) in the LMW Mor(Gly)₄-sKLH+aluminum group and were 143±1.23 (GMT±SD) in the LMW Mor(Gly)₄-sKLH+aluminum+INI-4001. Two-tailed unpaired t test with Welch's correction comparing titers between t showed a significant difference (p<0.01, t=4.008 df=9.655). Analysis of the serum and brain show a significantly increased serum concentration of 6-AM (FIG. 5B). No significant differences in brain 6-AM were detected, although a trending decrease can be seen (FIG. 5C). Heroin was not detected in the brain or the serum. These data suggest that the addition of INI-4001 to the Mor(Gly)₄-sKLH vaccine can increase antibody titers, which shifts the dose-response curve of heroin and further protects against its physiological effects.

“LMW Mor(Gly)₄-sKLH conjugate” and “Mor(Gly)₄-sKLH conjugate” are used interchangeably herein. In certain embodiments, a HMW Mor(Gly)₄-sKLH may be produced using the default protocol with the exception that the conjugate is also incubated at 37 °C following conjugation, prior to filtration, in an attempt to increase the size of the conjugate to produce HMW Mor(Gly)₄-sKLH. Unless specifically noted, Mor(Gly)₄-sKLH conjugate in the Examples does not refer to the HMW Mor(Gly)₄-sKLH, namely, Mor(Gly)₄-sKLH conjugate were made without incubation at 37 °C following conjugation. In certain embodiments, LMW Mor(Gly)₄-sKLH conjugate has a diameter of about 60-100 nm (e.g., 65-95 nm, 70-90 nm, or 75-85 nm) as determined by dynamic light scattering (DLS). In certain embodiments, HMW Mor(Gly)₄-sKLH conjugate has a diameter of about 150-250 nm (e.g., 160-240 nm, 170-230 nm, 180-220 nm or 190-210 nm) as determined by dynamic light scattering (DLS).

### Example 3

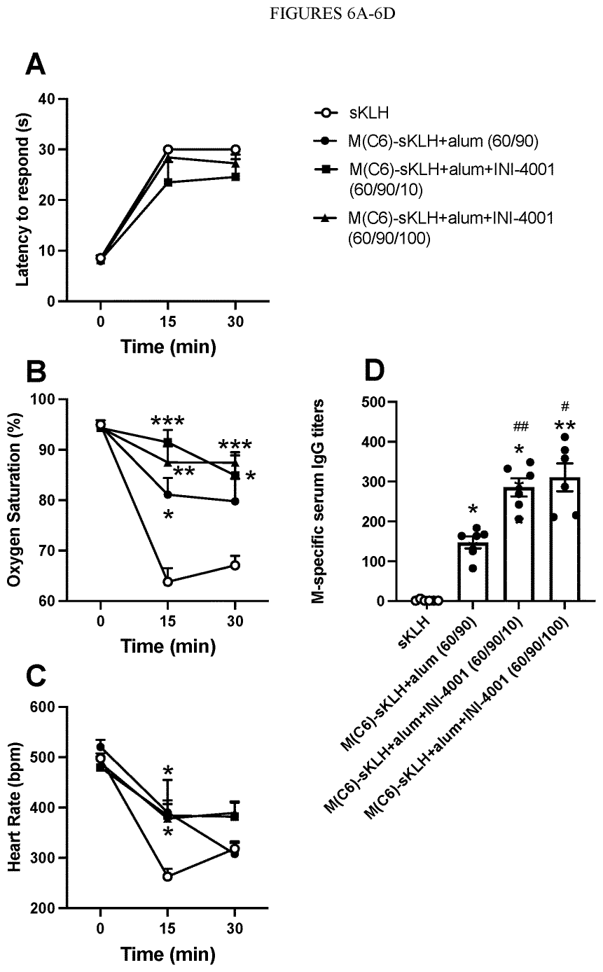

In this Example, the higher doses of M-sKLH, aluminum, and INI-4001 were used, e.g., to compare INI-4001 doses (10 or 100 μg formulations). Rats were immunized on days 0, 21, and 42 with sKLH as a negative control, 60 μg LMW Mor(Gly)₄-sKLH+90 μg aluminum, 60 g LMW Mor(Gly)₄-sKLH+90 μg aluminum+10 μg INI-4001, or 60 μg LMW Mor(Gly)₄-sKLH+90 μg aluminum+100 μg INI-4001 (FIG. 6). One week after the final immunization, morphine-specific serum IgG titers were assessed via ELISA. The following week, rats were challenged with 3 mg/kg heroin SC and rats were tested for antinociception on the hotplate and oximetry for oxygen saturation and heart rate. Mixed-effects ANOVA did not show differences between groups during antinociceptive testing for latency to respond (FIG. 6A), due to the large heroin dose and the control group reaching the maximum latency time (30 seconds). Oxygen saturation was protected in all groups 15 minutes after heroin dosing compared to controls, while only the INI-4001 containing vaccine formulations were protective 30 minutes later (FIG. 6B). Only the INI-4001 containing vaccine formulations were protective against heroin-induced bradycardia compared to controls at 15 minutes (FIG. 6C). Antibody titers were significantly increased in all vaccine formulations compared to sKLH controls, while both the low and high INI-4001 formulations showed significant increases in antibody titers compared to M-sKLH+aluminum alone. These data demonstrate that addition of INI-4001 may increase the efficacy of M-sKLH+aluminum, but that a high dose of INI-4001 might not be warranted as there was no benefit over the low INI-4001 containing formulation in the experimental setting of this Example.

### Example 4. Certain Exemplary Production of Conjugate and Vaccine Formulation

### Mor(Gly)₄-sKLH Drug Substance (DS)

The KLH carrier protein is supplied by Biosyn Corporation (Carlsbad, CA). Vacmune® is a subunit KLH (sKLH) formulation manufactured under GMP that is purified from the hemolymph of the sea snail, Meghathura crenulate.

Prior to conjugation, individual raw materials (i.e., hapten, cross-linker, and carrier protein) are filtered through a 0.20 μm filter and their concentrations measured before and after filtration by OD scans. The filter's membrane integrity is confirmed by bubble point testing. The hapten (5 mM) and N-ethyl-N′-(3 dimethylaminopropyl)carbodiimide hydrochloride (EDAC) cross-linker (208 mM) are dissolved in 0.1M 2-(N-morpholino)ethanesulfonic acid (MES) buffer at pH 5.0 and stirred for 10 minutes at room temperature. The sKLH solution is then added to the activated hapten and stirred for 3 hours at room temperature. The solution containing the conjugate mixture is then added to a Tangential Flow Filtration (TFF) holding reservoir and diafiltered with PBS, pH 7.2. The pH of the permeate is monitored and the diafiltration is stopped when the permeate reaches a pH of 7.0. The Mor(Gly)₄-sKLH conjugate drug substance cannot be sterile filtered as it is too large in diameter to pass through a 0.20 μm filter. The entire procedure is conducted under aseptic conditions using a laminar flow biosafety cabinet. The drug substance is stored at 2-8 °C.

### Example 5. Certain Exemplary Vaccine Formulation

### Mor(Gly)₄-sKLH Drug Product Manufacture

To formulate the drug product, equal volumes of 1600 μg/mL Mor(Gly)₄-sKLH drug substance in PBS, pH 7.2, 0.01% polysorbate 80 and 2400 μg/mL of aluminum (Alhydrogel 85 2%, Brenntag) in PBS 7.2, 0.01% polysorbate 80 are slowly mixed for a minimum of 30 minutes at room temperature. Bulk drug product is stored at 2-8 °C.

### Example 6. Certain Exemplary Vaccine Formulation

An aqueous formulation of INI-4001 (choline salt form) amenable to alum adsorption has been developed using an aqueous vehicle having 50 mM TRIS buffer at a pH of 7.4, 0.1% of tween-80 and 0.5% w/v monothioglycerol. The tween-80 may help obtain a colloidally stable formulation. Monothioglycerol (MTG) has been added to the formulation as an antioxidant because INI-4001 contains an oxidizable amine which can result in an oxidized degradant. An extensive antioxidant screen was conducted with INI-4001 and MTG was found to be the most effective at preventing the formation of the M+16 adduct. As described through this application, the resulting vaccine generates high titers of antibodies in rats that are highly specific for heroin and morphine.

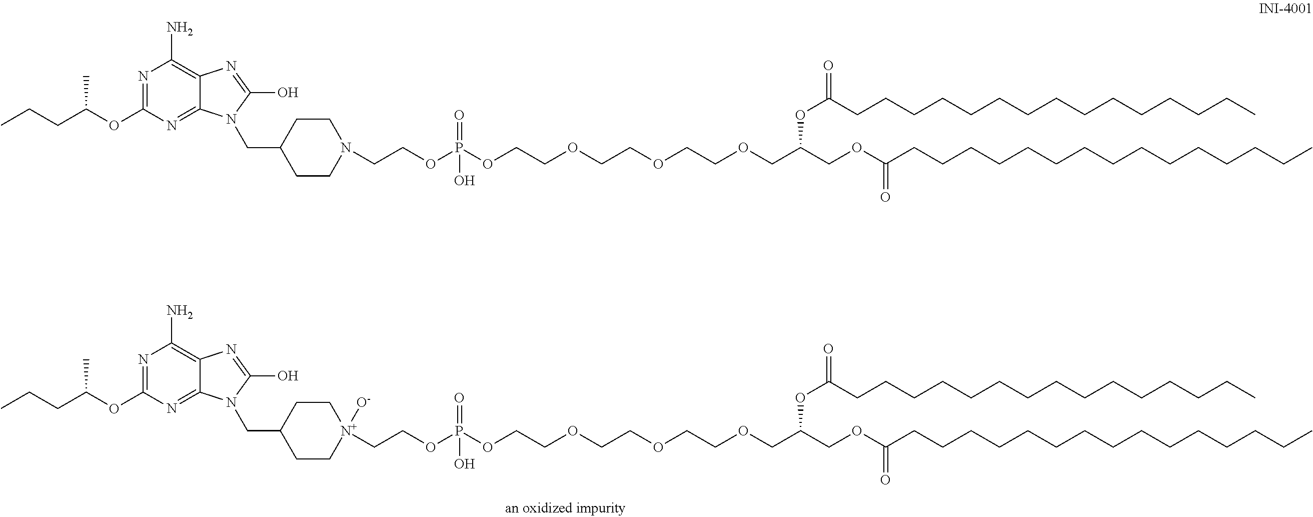

### Example 7. Toxicology Testing in Minipigs of a Heroin Vaccine Intended for Clinical Treatment of Opioid Use Disorder

A heroin vaccine (M-sKLH adsorbed to aluminum and adjuvanted with a TLR7/8 agonist INI-4001) is being developed for clinical evaluation to treat opioid use disorder (OUD) and has shown pre-clinical efficacy in rodents. Manufacturing of the heroin vaccine is underway, and the vaccine is currently undergoing toxicology assessment following biweekly injections (on Day 1, 15, 29, and 43) of low and high doses of vaccine (with and without the INI-4001 adjuvant) in male and female Gottingen minipigs (n=10/group). No adverse clinical signs and no dermal changes at the site of injection were observed following administration of all vaccine doses in any group. The highest morphine-specific serum IgG titers were observed in the group that received vaccine adjuvanted with INI-4001, and higher antibody titers were observed in the high vaccine dose group compared to the lower dose group. Group weights and food consumption did not differ between groups following administration of control or any vaccine group. These data demonstrate that M-sKLH adjuvanted with aluminum and INI-4001 is well tolerated and elicits high antibody levels in minipigs, suggesting that this vaccine formulation is safe for use in humans.

### Clinical Observation

There were no mortalities, no test items-related clinical observations, no changes at the dosing site (injection site observation) and no changes in body weights, food consumption, neurological evaluation, ophthalmoscopy, electrocardiography and clinical pathology parameters (hematology, coagulation, clinical chemistry and urinalysis). There were no test items-related organ weight and macroscopic findings.

Microscopic splenic, dosing site and/or mandibular lymph node findings were noted but overall, there were no effects of local (dosing site) or systemic toxicity.

In conclusion, the once every two weeks intramuscular injection of M-sKLH Conjugate Vaccine Adsorbed and INI-4001, when administered alone or in combination for 7 consecutive weeks (for a total of 4 doses) was well-tolerated with no overt signs of toxicity.

Based on parameters monitored on this study the no observable adverse effect level (NOAEL) was considered to be 400 μg/animal/dose for M-sKLH Conjugate Vaccine Adsorbed and 75 μg/animal/dose for INI-4001 when administered alone or in combination.

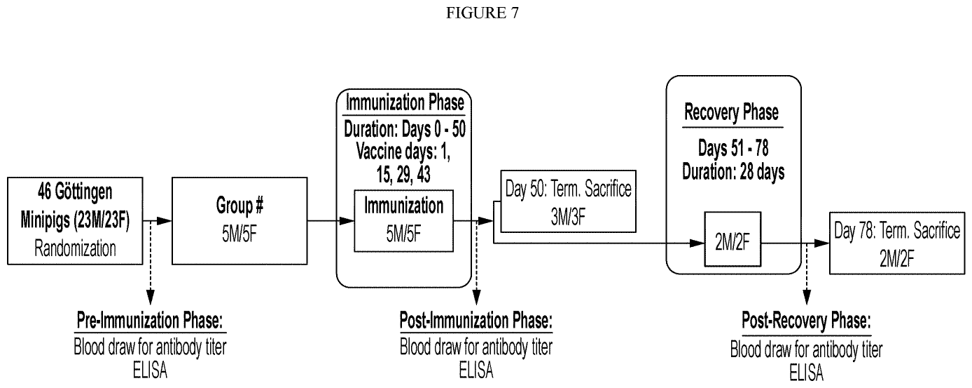

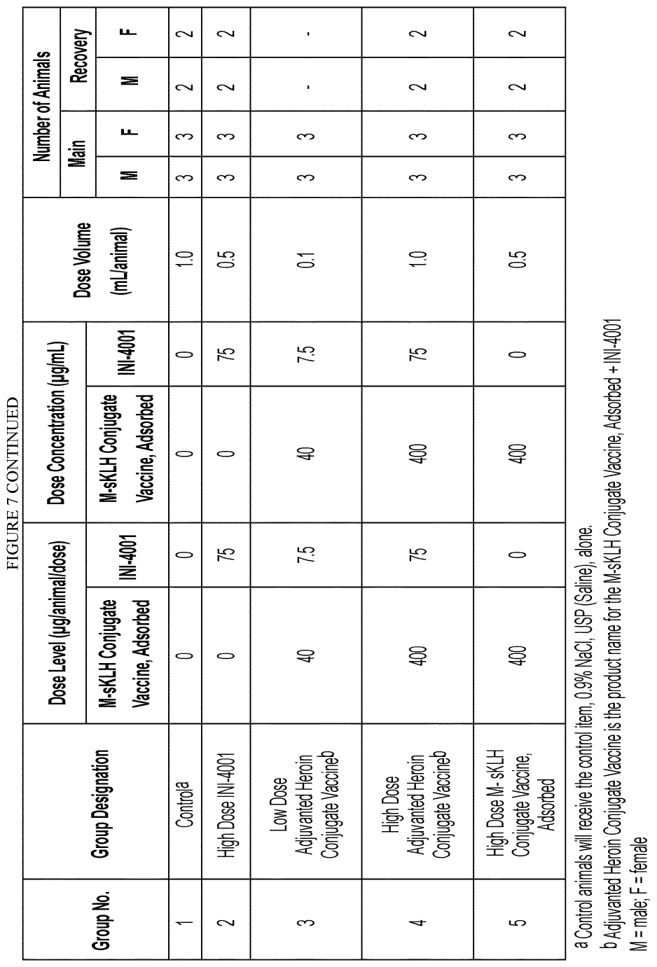

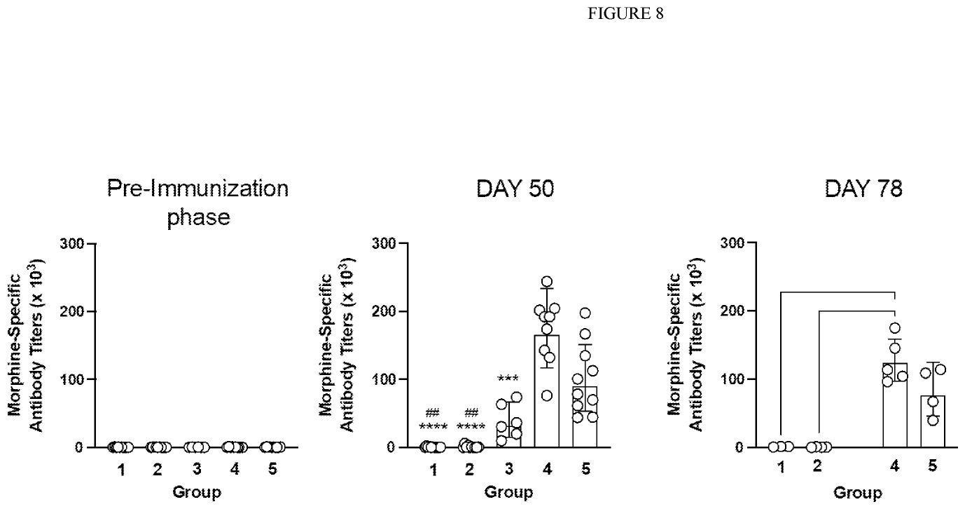

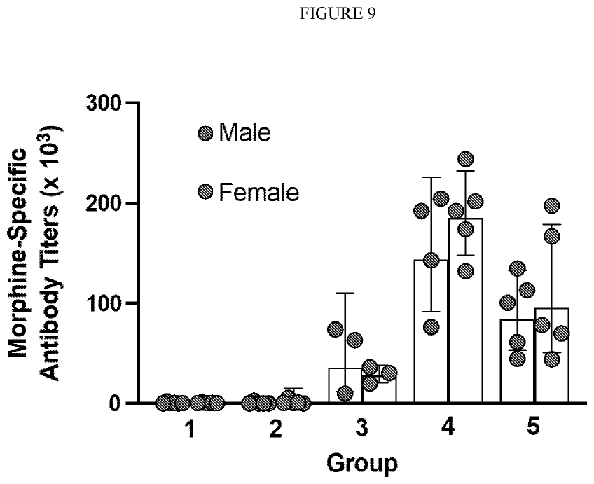

The experimental design, formulations, and results are also shown in FIG. 7, FIG. 8, and FIG. 9, and brief description of FIGS. 7-9.

### REFERENCES CITED IN EXAMPLE 7

Baruffaldi F, Raleigh M D, King S J, Roslawski M J, Birnbaum A K, Hassler C, Carroll F I, Runyon S P, Winston S, Pentel P R, Pravetoni M. Formulation and characterization of conjugate vaccines to reduce opioid use disorders suitable for pharmaceutical manufacturing and clinical evaluation. Mol Pharm. 2019 Jun. 3; 16(6):2364-2375.
Alyson J. Smith, Yufeng Li, Hélène G. Bazin, Julien R. St-Jean, Daniel Larocque, Jay T. Evans, and Jory R. Baldridge Evaluation of novel synthetic TLR7/8 agonists as vaccine adjuvants. Vaccine. 2016 Aug. 5; 34(36): 4304-4312. doi:10.1016/j.vaccine.2016.06.080
Raleigh M D, Laudenbach M, Baruffaldi F, Peterson S J, Roslawski M J, Birnbaum A K, Carroll F I, Runyon S P, Winston S, Pentel P R, Pravetoni M. Opioid dose-and route-dependent efficacy of oxycodone and heroin vaccines in rats. Journal of Pharmacology and Experimental Therapeutics. 2018 May; 365(2):346-353
Raleigh M D, Pentel P R, LeSage M G. Pharmacokinetic correlates of the effects of a heroin vaccine on heroin self-administration in rats. PloS One. 2014 Dec. 23; 9(12):el 15696.
Raleigh M D, Pravetoni M, Harris A C, Birnbaum A K, Pentel P R. Selective effects of a morphine conjugate vaccine on heroin and metabolite distribution and heroin-induced behaviors in rats. Journal of Pharmacology and Experimental Therapeutics. 2013 February; 344:397-406.

### Example 9. Certain Exemplary Vaccine Formulation

The composition of the Drug Product (DP) comprises 0.8 mg/ml Mor(Gly)₄-sKLH, 0.02% (0.2 mg/ml) Polysorbate 80 (PS80), and 1.2 mg/ml Aluminum in PBS pH 7.2. This will be mixed 1:1 with an aqueous solution comprising INI-4001 (e.g., see Example 6).

After mixing, the composition comprises 0.4 mg/ml Mor(Gly)₄-sKLH, and 0.6 mg/ml Aluminum.

### Example 10. Exemplary Synthesis Route for Production of a Morphine Hapten or Salt Thereof

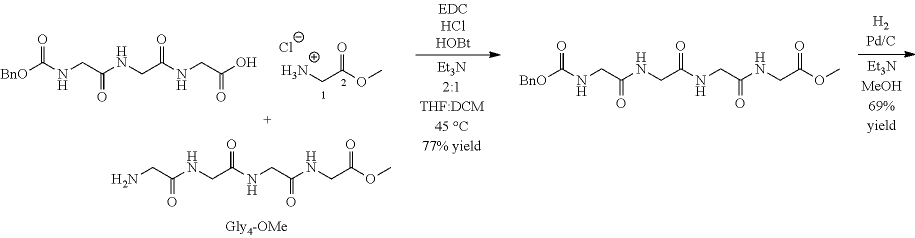

### Scheme 1: Generation of the Tetraglycine Linker Gly₄OMe

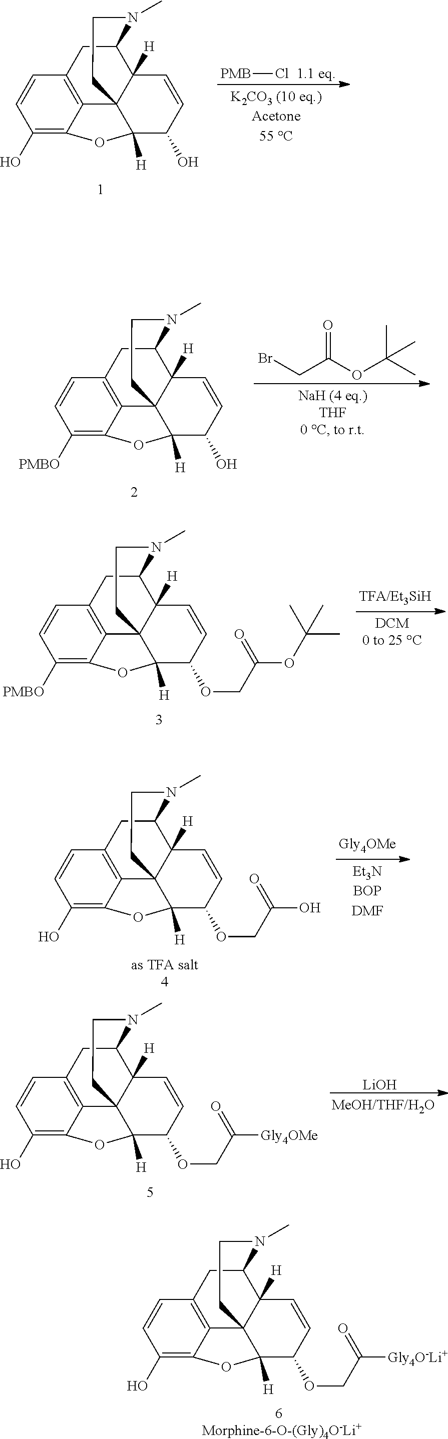

### Scheme 2: Synthetic Route Used to Produce Morphine-6-O-(Gly)₄O-Li+

### Step 1: Para-Methoxybenzyl (PMB) Protection of Phenol Hydroxy of Morphine

Protection of the phenol hydroxyl group utilizes para-methoxybenzyl (PMB) protection conditions. This involved using PMB-chloride and potassium carbonate in near refluxing acetone.

Procedure (Step 1): A 12-L Morton flask was equipped with overhead, mechanical stirrer, reflux condenser, thermocouple, and N₂(g) inlet. The flask was charged with acetone (4.5 L, 18V) and morphine hemi-sulfate pentahydrate dimer (250 g, 660 mmol) followed by an acetone rinse (0.5 L, 2V). To this white slurry, potassium carbonate was added (914 g, 6.6 mmol, 10 eq), followed by para-methoxybenzyl chloride (70 mL, 515 mmol, 0.78 eq). The reaction mixture was heated to 55 °C for 27 hours. The reaction stirred at 20 °C for an additional 48 hours. An additional volume of para-methoxybenzyl chloride added (30 mL, 220 mmol, 0.33 eq) and the reaction mixture was heated to 55 °C for 16 hours. A final aliquot of para-methoxybenzyl chloride added (7.2 mL, 53 mmol, 0.08 eq) and the reaction mixture stirred for an additional 16 hours.

The reaction mixture was concentrated to ~1.5 L of total volume and diluted with DI water (5.0 L, 20V). Extract with 9:1 dichloromethane:methanol (2×2.5 L, 2×10V). The organic layers were combined and evaporate to dryness. The crude was dissolved in dichloromethane (900 mL, 3.6V) and prefiltered before loading onto 3.0 kg ISCO column preequilibrated with dichloromethane.

The column purification mobile phases are as follows: Solvent A: DCM; Solvent B: 90:9:1 DCM:MeOH:NH₄OH. The column purification conditions listed below (table 1).

TABLE 1
(Purification conditions for step 1).

| | Column |
|---|---|
| % Solvent B | Volumes |
| 0-60% | 20 CV |
| 60% | 5 CV |
| 60-80% | 10 CV |
| 80% | 5 CV |

Select fractions were analyzed by HPLC to determine the AUC % purity at different intervals. The fractions were separated into two lots, either clean or contaminated with bis-PMB byproduct. The impure batches were repurified using the same conditions until sufficient quantity of pure product was isolated. Four column purifications were utilized to isolate a total of 117 g (290 mmol, 43% yield) of (4R,4aR,7S,7aR,12bS)-9-((4-methoxybenzyl)oxy)-3-methyl-2,3,4,4a,7,7a-hexahydro-1H-4,12-methanobenzofuro[3,2-e]isoquinolin-7-ol (compound 2); ¹H NMR (400 MHz, CHLOROFORM-d) 6=7.38-7.28 (m, 2H), 6.95-6.83 (m, 2H), 6.75-6.66 (m, 1H), 6.57-6.47 (m, 1H), 5.76-5.58 (m, 1H), 5.28-5.20 (m, 1H), 5.12-4.95 (m, 2H), 4.92-4.79 (m, 1H), 4.23-4.06 (m, 1H), 3.85-3.73 (m, 3H), 3.53-3.31 (m, 1H), 3.09-2.97 (m, 1H), 2.90-2.63 (m, 3H), 2.59-2.43 (m, 4H), 2.42-2.29 (m, 1H), 2.25-2.08 (m, 1H), 1.94-1.76 (m, 1H).

### Step 2: Alkylation of Compound 2 Using Tert-Butyl Bromo Acetate to Prepare Compound 3.

Preparation of tert-butyl 2-(((4R,4aR,7S,7aR,12bS)-9-((4-methoxybenzyl)oxy)-3-methyl-2,3,4,4a,7,7a-hexahydro-1H-4,12-methanobenzofuro[3,2-e]isoquinolin-7-yl)oxy)acetate (compound 3) was performed by deprotonation of the alcohol using sodium hydride followed by alkylation with tert-butyl bromo acetate. The reagents were to be charged at −10 °C to lower the energy of the exotherm that occurs during the addition of both the sodium hydride and alkylating agent.

Procedure (Step 2): A 3-L jacketed 3-neck round bottom flask was equipped with overhead, mechanical stirrer, thermocouple, and N₂(g) inlet. The reactor was charged with compound 2 (116 g) dissolved in tetrahydrofuran (0.93 L, 8V). The reaction was cooled to −4.5 °C and sodium hydride (60% in mineral oil, 35 g, 4.0 eq) were added portion wise to the solution over a 22-minute period. An additional volume of tetrahydrofuran (0.09 L, 2V) was used to rinse the sodium hydride from the funnel to the reaction mixture. The reaction was aged for 15 minutes and a slight exotherm of 1.5 °C was observed. The reaction temperature equilibrated down to −3.3 °C and tert-butyl bromo acetate was then charged to the reaction mixture over a period of 7 minutes with an exotherm to 0.8 °C being observed. The resulting temperature of 0.8 °C was then allowed to equilibrate to −5.7 °C while aging for 16 hours.

The reaction was quenched by charging aqueous saturated ammonium chloride (0.93 L, 8V) over a 1-hour period. An exotherm from −4.2 °C to 14 °C was observed. The resulting aqueous solution was extracted with dichloromethane (3×930 mL, 3×8V). Prefilter and separate into two lots before loading onto 2 separate 1.5 kg ISCO column preequilibrated with dichloromethane.

The column purification mobile phases are as follows: Solvent A: DCM; Solvent B: 90:9:1 DCM:MeOH:NH₄OH. The column purification conditions listed below (table 2).

TABLE 2
(Purification conditions for step 2).

| | Column |
|---|---|
| % Solvent B | Volumes |
| 0-40% | 20 CV |
| 40% | 5 CV |
| 40-80% | 10 CV |
| 80% | 5 CV |

The fractions were analyzed by HPLC to determine the AUC % purity at different intervals. The fractions were separated into two lots, either clean or new unknown byproduct. Two column purifications were required to isolate a total of 73 g (140 mmol, 67% yield) of compound 3; ¹H NMR (400 MHz, CHLOROFORM-d) 6=7.42-7.33 (m, 2H), 6.94-6.77 (m, 2H), 6.68-6.57 (m, 1H), 6.51-6.39 (m, 1H), 5.89-5.73 (m, 1H), 5.36-5.31 (m, 1H), 5.14-5.08 (m, 2H), 5.05-4.99 (m, 1H), 4.30-4.15 (m, 2H), 4.13-4.06 (m, 1H), 3.84-3.72 (m, 3H), 3.55-3.38 (m, 1H), 3.12-2.98 (m, 1H), 2.95-2.08 (m, 9H), 2.01-1.87 (m, 1H), 1.54-1.42 (m, 9H).

### Step 3: Global Deprotection of the PMB and Tert-Butyl Groups in 3 Using TFA and TES to Prepare Compound 4.

Preparation of 2-(((4R,4aR,7S,7aR,12bS)-9-hydroxy-3-methyl-2,3,4,4a,7,7a-hexahydro-1H-4,12-methanobenzofuro[3,2-e]isoquinolin-7-yl)oxy)acetic acid (compound 4) uses a mixture of triethyl silane (TES) and trifluoracetic acid (TFA) to globally deprotect both the para-methoxybenzyl group as well as the tert-butyl group. The addition of trifluoroacetic acid is exothermic and requires the use of cool temperatures during addition.

Procedure (Step 3): A 3-L jacketed 3-neck round bottom flask was equipped with overhead, mechanical stirrer, thermocouple, and N₂(g) inlet. The reactor was charged with compound 3 (76 g) in dichloromethane (1.46 L, 20V). The solution was cooled to 0 °C and charged with triethyl silane (0.34 L, 15.0 eq) in one batch. To this solution, trifluoroacetic acid (0.22 L, 20 eq) wash charged over a period of 20 minutes. A small exotherm of 4.2 °C was observed. The reaction mixture was slowly warmed to 25 °C and stirred for a period of 24 hours. The organic solvents were then evaporated en vacuo. To the resulting thin oil, heptane (0.73 L, 10V) was added to the material and used to azeotrope off any remaining organic liquids. The crude material was then dissolved in acetonitrile (0.73 L, 10V) and evaporated en vacuo to deliver a crude oil.

The crude was partitioned between acetonitrile (0.73 L, 10V) and heptane (0.73 L, 10V), with the lower acetonitrile layer being isolated and concentrated to an oil. The resulting oil was dissolved in acetonitrile (0.36 L, 5V) and toluene (0.73 L, 10V) and concentrated to an oil. The crude oil was again brought up in acetonitrile (0.36 L, 5V, then 0.18 L, 2.5V) and concentrated to an oil after each addition of acetonitrile to yield 71 g (>theoretical) of compound 4 as a foam; ¹H NMR (400 MHz, DMSO-d6) δ=10.26-9.93 (m, 1H), 7.08-6.96 (m, 1H), 6.83-6.76 (m, 1H), 6.59-6.50 (m, 1H), 6.48-6.37 (m, 1H), 5.80-5.68 (m, 1H), 5.40-5.23 (m, 1H), 5.14-5.05 (m, 1H), 5.05-4.98 (m, 1H), 4.22-4.15 (m, 3H), 4.14-3.97 (m, 3H), 3.36-2.61 (m, 11H), 2.26-2.11 (m, 2H), 2.07-1.99 (m, 4H), 1.96-1.86 (m, 1H).

Step 4: BOP Amide Coupling of Compound 4 with Gly₄OMe to Produce Compound 5.

Procedure (Step 4): A 3-L jacketed 3-neck round bottom flask was equipped with overhead, mechanical stirrer, thermocouple, and N₂(g) inlet. The reactor was charged with compound 4 (68 g) in dimethylformamide (0.54 L, 8 V). Gly₄OMe (43 g, 1.1 eq) was added to the solution, which was then cooled to 0 °C. Benzotriazol-1-yloxytris(dimethylamino)phosphonium hexafluorophosphate (BOP) (86 g, 1.3 eq) and triethyl amine (TEA) (83 mL, 4 eq) were added to the reaction mixture sequentially, which was warmed to 25 °C and aged for 17 hours.

The solvent was concentrated to around half volume and dried onto celite (500 g). The material was split into four lots and purified using the following mobile phase conditions: Solvent A: DCM; Solvent B: 90:9:1 DCM:MeOH:NH₄OH (table 3).

TABLE 3
(Purification conditions for step 4).

| | Column |
|---|---|
| % Solvent B | Volumes |
| 0-100% | 45 CV |
| 100% | 25 CV |

An aliquot was taken from select product fractions to dry down an analyze by ¹H NMR (MeOD) to determine which fractions were of sufficient quality to combine. The clean fractions were combined and concentrated to dryness to yield 50 g (57% yield over two steps) of methyl (2-(((4R,4aR,7S,7aR,12bS)-9-hydroxy-3-methyl-2,3,4,4a,7,7a-hexahydro-1H-4,12-methanobenzofuro[3,2-e]isoquinolin-7-yl)oxy)acetyl)glycylglycylglycylglycinate (compound 5); ¹H NMR (400 MHz, DMSO-d6) δ=9.12-8.78 (m, 1H), 8.31-8.23 (m, 1H), 8.22-8.12 (m, 2H), 8.06-7.90 (m, 1H), 6.56-6.46 (m, 1H), 6.44-6.29 (m, 1H), 5.82-5.67 (m, 1H), 5.42-5.30 (m, 1H), 5.12-4.97 (m, 1H), 4.19-4.03 (m, 4H), 3.92-3.79 (m, 4H), 3.79-3.71 (m, 4H), 3.67-3.60 (m, 3H), 3.20-3.12 (m, 2H), 3.06-2.92 (m, 1H), 2.76-2.54 (m, 3H), 2.47-2.18 (m, 2H), 2.16-1.98 (m, 1H), 1.83-1.64 (m, 1H).

Step 5: Hydrolysis of Ester of Compound 5 to Produce Morphine-6-O-(Gly)₄O—Li⁺.

Procedure (Step 5): A 3-L 3-neck round bottom flask was equipped with overhead, mechanical stirrer, thermocouple, and N₂(g) inlet. The reactor was charged with compound 5 (49 g) in 1:1:1 methanol:tetrahydrofuran:water (0.98 L, 20V). To this cloudy solution, lithium hydroxide monohydrate (3.7 g, 1.1 eq) was added in one batch. The reaction was aged at 25 °C for 24 hours. Additional lithium hydroxide monohydrate (0.37 g, 0.1 eq) was added and aged for an additional 71 hours. The solution was concentrated to 1/3 volume to remove most of the volatile organics. The solution was diluted with acetonitrile (0.33 L, 6.7V) and frozen in dry ice/acetone bath. The solid solution was then place on lyophilizer for 46 hours to yield 46 g (96%) of Morphine-6-O-(Gly)₄O⁻Li⁺; ¹H NMR (400 MHz, METHANOL-d4) δ=8.00-7.95 (m, 1H), 6.60-6.53 (m, 1H), 6.48-6.41 (m, 1H), 5.80-5.70 (m, 1H), 5.47-5.36 (m, 1H), 5.12-5.03 (m, 1H), 4.37-4.18 (m, 2H), 4.18-4.12 (m, 1H), 4.07-3.99 (m, 2H), 3.98-3.92 (m, 2H), 3.91-3.86 (m, 2H), 3.81-3.71 (m, 2H), 3.43-3.36 (m, 1H), 3.10-3.00 (m, 1H), 3.00-2.98 (m, 1H), 2.87-2.85 (m, 1H), 2.73-2.56 (m, 2H), 2.51-2.40 (m, 4H), 2.40-2.30 (m, 1H), 2.17-2.02 (m, 1H), 1.90-1.79 (m, 1H).

### Example 11. Effects of M-sKLH/Alum and M-sKLH/Alum+INI-4001 in the Context of Methadone Maintenance

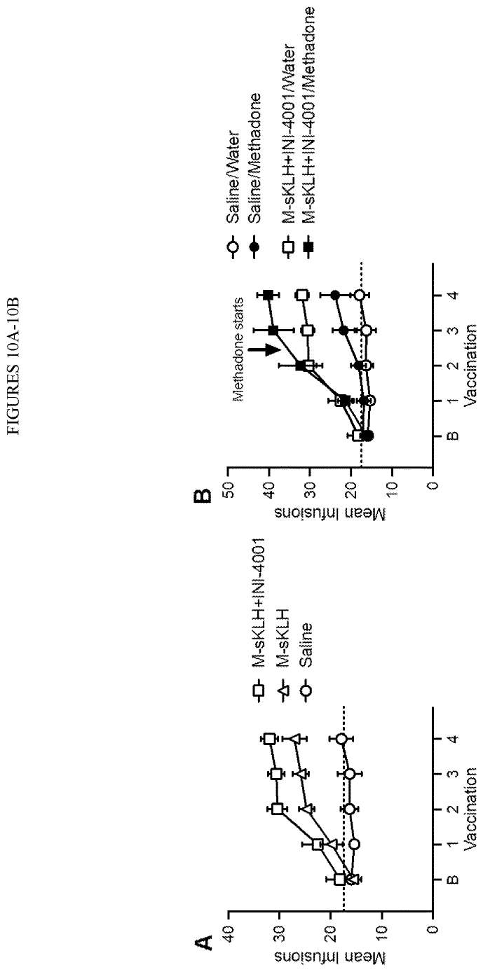

Five groups of rats have been trained to intravenously self-administer 0.06 mg/kg heroin. Once heroin self-administration (HSA) is stable, rats were vaccinated every two weeks with M-sKLH+INI-4001, M-sKLH, or saline (control). One week after the second vaccination, M-sKLH+INI-4001 and saline control rats were implanted with an osmotic minipump to deliver a continuous s.c. infusion of either sterile water (control) or 20 mg/kg/day methadone. Rats vaccinated with M-sKLH were only implanted with a water pump to allow the effect of INI-4001 on M-sKLH efficacy to be determined. Rats continue biweekly vaccinations during the methadone infusion for another 8 weeks. During the last 4 weeks methadone maintenance, the heroin dose was reduced weekly to obtain a dose-response curve (0, 0.003, 0.01, 0.03 mg/kg/inf). Preliminary data show that both vaccines attenuate the reinforcing effects of heroin as indicated by a compensatory increase in HSA, with a trend for M-sKLH+INI-4001 to produce a larger effect than M-sKLH (FIG. 10A). In addition, the combination of M-sKLH+INI-4001 with methadone produces the greatest increase in HSA compared to either M-sKLH+INI-4001 or methadone alone (FIG. 10B). Finally, relatively few rats have completed the HSA dose-response assessment. However, data thus far suggest the heroin dose-response curve is shifted to the right by M-sKLH+INI-4001 and slightly further still by M-sKLH+INI-4001 during methadone infusion, indicating a decrease in heroin potency (data not shown). Together these findings show that INI-4001 improves the efficacy of M-sKLH at attenuating the reinforcing potency of heroin, and methadone maintenance further enhances this effect.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

What is claimed is:

1. A vaccine composition comprising one or more adjuvants (e.g., two adjuvants), and
a morphine-carrier protein conjugate having structure of Formula I: 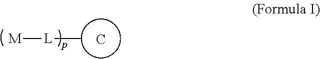
wherein
L is a linker,
C is a carrier protein,
p is a hapten to carrier protein ratio number that is >=1, and
M has structure of 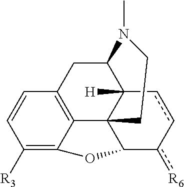
wherein R₃ is OH and R₆ is O or N that is bonded with L; or
R₆ is OH and R₃ is O that is bonded with L.

2. The vaccine composition of claim 1, wherein the one or more adjuvants comprises aluminum.

3. The vaccine composition of any one of claims 1-2, wherein the one or more adjuvants comprises aluminum hydroxide.

4. The vaccine composition of any one of claims 1-3, wherein the one or more adjuvants comprises a TLR7/8 agonist.

5. The vaccine composition of any one of claims 1-4, wherein the one or more adjuvants comprises INI-4001 having structure of: 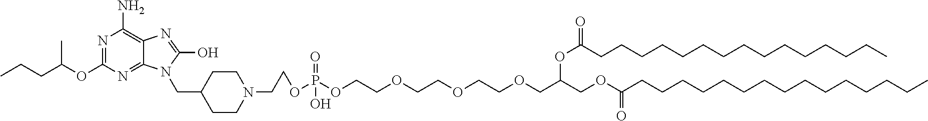 [SMILES: CCCCCCCCCCCCCCCC(=O)OCC(COCCOCCOCCOP(=O)(O)OCCN1CCC(Cn2c(O)nc3c(N)nc(OC(C)CCC)nc32)CC1)OC(=O)CCCCCCCCCCCCCCC]
or salt thereof.

6. The vaccine composition of any one of claims 1-5, wherein the vaccine composition comprises morphine-carrier protein conjugate and aluminum that are at a weight ratio of about 1:1, 2:3, 1:2, 1:3, 1:4, or 1:5.

7. The vaccine composition of any one of claims 1-6, wherein the vaccine composition comprises morphine-carrier protein conjugate and aluminum that are at a weight ratio of about 1:1 to 1:5.

8. The vaccine composition of any one of claims 1-7, wherein the vaccine composition comprises morphine-carrier protein conjugate and aluminum that are at a weight ratio of about 2:3 to 1:3 (e.g., about 2:3).

9. The vaccine composition of any one of claims 1-8, wherein the vaccine composition comprises morphine-carrier protein conjugate and INI-4001 that are at a weight ratio of about 1:2, 3:5, 1:1, 2:1, 3:1, 4:1, 5:1, or 6:1.

10. The vaccine composition of any one of claims 1-9, wherein the vaccine composition comprises morphine-carrier protein conjugate and INI-4001 that are at a weight ratio of about 1:2 to 6:1.

11. The vaccine composition of any one of claims 1-10, wherein the vaccine composition comprises morphine-carrier protein conjugate and INI-4001 that are at a weight ratio of about 5:1 to 6:1 (e.g., about 5.3:1).

12. The vaccine composition of any one of claims 1-11, wherein the vaccine composition comprises morphine-carrier protein conjugate, aluminum, and INI-4001 that are at a weight ratio of about 6:9:1, or 5.3:8:1, or 1:5:2.

13. The vaccine composition of any one of claims 1-12, wherein the vaccine composition comprises about 10-800 μg morphine-carrier protein conjugate (e.g., 40, or 400 μg).

14. The vaccine composition of any one of claims 1-13, wherein the vaccine composition comprises about 15-1200 μg aluminum (e.g., 60, or 600 μg).

15. The vaccine composition of any one of claims 1-14, wherein the vaccine composition comprises about 1.8-150 μg INI-4001 (e.g., 7.5, or 75 μg).

16. The vaccine composition of any one of claims 1-15, wherein the vaccine composition further comprises an antioxidant (e.g., monothioglycerol (MTG)).

17. The vaccine composition of any one of claims 1-16, wherein the composition does not comprise a morphine-carrier protein conjugate according to claim 1 wherein R₆ is OH and R₃ is O that is bonded with L.

18. The vaccine composition of any one of claims 1-17, wherein M has structure of Formula Ia:

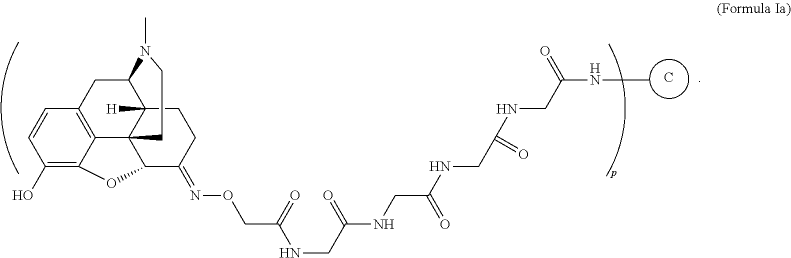

19. The vaccine composition of any one of claims 1-17, wherein M has structure of Formula Ib:

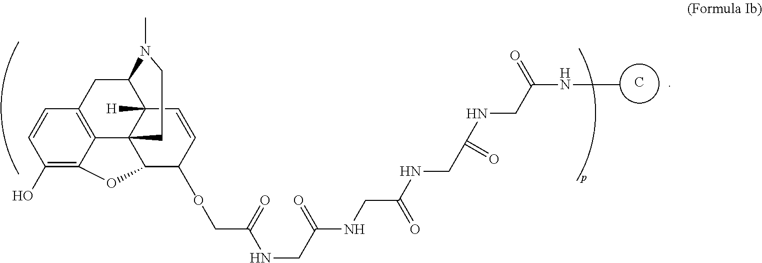

20. The vaccine composition of any one of claims 1-19, wherein C is Keyhole limpet hemocyanin.

21. The vaccine composition of any one of claims 1-20, wherein C is subunit KLH (sKLH).

22. The vaccine composition of any one of claims 1-21, which is capable of protecting a subject immunized with the vaccine composition against heroin-induced respiratory depression.

23. The vaccine composition of any one of claims 1-22, which is capable of protecting a subject immunized with the vaccine composition against heroin-induced bradycardia.

24. The vaccine composition of any one of claims 1-23, which is capable of sequestering heroin in the blood of the subject immunized with the vaccine composition, who has a serum heroin concentration that is at least 15% (e.g., 20%, 30%, 40%, 50%, 60%, 70%, 80%, 90%, 100%, 150%, 200% or more) higher as compared to a control (e.g., receiving same vaccine without TLR7/8 agonist, such as INI-4001).

25. A method of treating opioid use disorder (OUD) or immunizing a subject (e.g., for preventing opioid overdose), comprising administering a vaccine composition according to any one of claims 1-24 to the subject.

26. The method of claim 25, wherein heroin specific antibody is induced in the subject.

27. The method of claim 25 or 26, wherein the subject is protected against heroin-induced bradycardia.

28. The method of any one of claims 25-27, wherein the subject is protected against heroin-induced respiratory depression.

29. The method of any one of claims 25-28, further comprising administering an additional OUD therapeutic agent to the subject.

30. The method of claim 29, wherein the additional OUD therapeutic agent is methadone.

31. The method of any one of claims 25-30, wherein the subject is protected against heroin self-administration.

32. A method, comprising converting morphine (compound 1) to (4R,4aR,7S,7aR,12bS)-9-((4-methoxybenzyl)oxy)-3-methyl-2,3,4,4a,7,7a-hexahydro-1H-4,12-methanobenzofuro[3,2-e]isoquinolin-7-ol (compound 2).

33. The method of claim 32, comprises reacting morphine (compound 1) with para-methoxybenzyl chloride.

34. The method of claims 32 or 33, further comprising converting compound 2 to compound 3.

35. The method of claim 34, wherein the converting compound 2 to compound 3 comprises reacting compound 2 with tert-butyl bromo acetate (e.g., contacting compound 2 with NaH and tert-butyl bromo acetate).

36. The method of any one of claims 32-35, further comprising converting compound 3 to compound 4.

37. The method of claim 36, wherein the converting compound 3 to compound 4 comprises contacting compound 3 with trifluoracetic acid (TFA) in the presence of triethyl silane (TES).
